(19) United States
(12) Patent Application Publication
(10) Pub. No.: US 2025/0169735 A1
(43) Pub. Date: May 29, 2025

(54) RADIOGRAPHY-CONCURRENT DYNAMIC ELECTROPOTENTIAL NEUROACTIVITY MONITOR

(71) Applicant: ASTERION AI INC., Dallas, TX (US)

(72) Inventors: Ezekiel Fink, Dallas, TX (US); Kenneth Liu, Kalamazoo, MI (US); Krag Browder, Colleyville, TX (US); Leme Mohamed, Dallas, TX (US); Hannah Olander, Dallas, TX (US); Jeff Young, Dallas, TX (US); Milcah Taye, Dallas, TX (US); Teresa Thai, Dallas, TX (US); Willam Peterson, Dallas, TX (US); Nithya Ramakrishnan, Dallas, TX (US)

(73) Assignee: ASTERION AI INC., Dallas, TX (US)

(21) Appl. No.: 18/866,953

(22) PCT Filed: May 19, 2023
(86) PCT No.: PCT/US2023/022895

§ 371 Date: Nov. 18, 2024

Related U.S. Application Data
(60) Provisional application No. 63/386,693, filed on Dec. 9, 2022.
(60) Provisional application No. 63/365,003, filed on May 19, 2022.

(51) Int. Cl. A61B 5/291; A61B 5/256; A61B 6/46; A61B 6/50

(52) U.S. Cl. CPC A61B 5/291; A61B 5/256; A61B 6/461; A61B 6/501

(57) ABSTRACT

Apparatus and associated methods relate to assisting gait impaired patients. In an illustrative example, a gait assisting apparatus may be wearable by a user including a sensor module and an actuator. The sensor module may be configured to generate a sensor measurement from measured data associated with the user. For example, a controller operably coupled to the sensor module may include a local classification model configured to classify a gait situation based on a classification input received from the sensor module. In some implementations, an activation module of the controller may generate an activation level to control the actuator. In operation, the activation module may apply the local classification model to the classification input to determine the activation level of the actuator to generate a vibration gait assistance and/or illumination guidance. Various embodiments may advantageously provide a gait assistant function to prevent gait impairment injuries.

### CROSS-REFERENCE TO RELATED APPLICATIONS

This application claims the benefit of U.S. Provisional Application Ser. No. 63/386,693,titled “Field EEG Stroke Progression Detection,” filed by Ezekiel Fink, on Dec. 9, 2022; and U.S. Provisional Application Ser. No. 63/365,003, titled “Realtime Cross-Modality Spatial Brain Activity Intervention Guidance,” filed by Ezekiel Fink, et al., on May 19, 2022.

This application incorporates the entire contents of the foregoing application(s) herein by reference.

The subject matter of this application may have common inventorship with and/or may be related to the subject matter of the following:
PCT Patent Application Serial No. PCT/US2023/060120, titled “Stroke Prediction Multi-Architecture Stacked Ensemble Supermodel,” filed by Ezekiel Fink, et. al., on Jan. 4, 2023.

This application incorporates the entire contents of the foregoing application(s) herein by reference.

### TECHNICAL FIELD

Various embodiments relate generally to generating visual guidance related to real-time brain activity.

### BACKGROUND

An electroencephalogram (EEG) is a noninvasive tool that records the electrical activity of the brain. For example, an EEG may be performed by placing several electrodes on the scalp that are sensitive to small electrical changes to detect brain cell activity. EEG signals, for example, may be amplified, filtered, and graphed for analysis. On an EEG graph, for example, an x-axis may represent time, and a y-axis may represent a voltage of the brain activity. Readings from the individual electrodes may be displayed separately in rows in a referential montage formation.

Fluoroscopy is a medical procedure that makes a real-time video of the movements inside a part of a body (e.g., a brain) by passing x-rays through the body over a period of time. In some examples, fluoroscopy may be used for diagnosing various health problems, including heart, intestinal, and/or brain disease. In some cases, fluoroscopy may be used to guide treatments such as implants or injections, or in surgery operations. For example, a healthcare provider may use fluoroscopy to look inside organs, joints, muscles, and/or bones.

European Patent Application Publication Serial No. EP 3 235 426 filed by Gary Loubert, et al., discloses a device for performing EEG consisting an elastic head strap. Japan Patent Serial No. JP 6,927,632 B2 filed by Nitta Corp., discloses an electrode for measuring electroencephalogram.

### SUMMARY

Apparatus and associated methods relate to a signal acquisition cap. In an illustrative example, an EEG cap may include a soft support structure and a clip-on electrode unit. The soft support structure, for example, may include elastic bands in a lattice formation. The clip-on electrode unit may be releasably coupled, for example, to the soft support structure. The clip-on electrode unit may include an electrode body of radiotranslucent materials. A conductive coating, for example, may be disposed on a surface of the electrode body such that the clip-on electrode unit is substantially radiotranslucent. For example, when the clip-on electrode unit is deployed on a skin surface, the spring-loaded recording channel is configured to conduct EEG signals from the skin surface to a remote computer system. Various embodiments may advantageously deploy the EEG cap without obstructing a view of a concurrently operating radioactivity imaging tool.

Apparatus and associated methods relate to a signal visualization system (SVS). In an illustrative example, a SVS may include a radiotranslucent electrode cap configured to detect EEG signals. For example, the electrode cap may be effectively radiotranslucent in a radiographic image. For example, the SVS may include a computer system coupled to the radiotranslucent electrode cap to receive the EEG signals. For example, the computer system may include an EEG features generation engine, a brain health classification model, and a display engine. In operation, the EEG features generation engine may generate, in near real-time, EEG features as a function of EEG signals received from the radiotranslucent electrode cap. For example, the EEG features may be applied to the brain health classification model to generate a brain health indicia to be displayed by the display engine. Various embodiments may advantageously display a real-time visual indicia relating to a brain health.

Apparatus and associated methods relate to systems and methods to display brain activity from multiple live data feeds. In an illustrative example, an exemplary Brain Activity Monitoring System (BAMS) may include an EEG cap having radio translucent leads, and radiography equipment for monitoring a patient during a surgery operation. The EEG cap, for example, may be connected to a relocatable EEG control unit. The BAMS may generate visual images using live data from the EEG cap and the radiography equipment, for example. In some implementations, the visual images may be two-dimensional. In some implementations, the visual images may be a three-dimensional (3D) topology. In some implementations, the BAMS may determine an abnormality in the brain by comparing the live data and target images using a classification model. Various embodiments may advantageously generate a display to provide real-time guidance of brain activity during the surgery operation.

Various embodiments may achieve one or more advantages. For example, some embodiments may include a chin strap and intersecting modules to advantageously allow size adjustment of the cap. Some embodiments, for example, may include a remotely placed amplifying circuit to advantageously avoid obstruction of radiography of a body. For example, some embodiments may advantageously display a temporal distribution, a spatial distribution, and/or a spectral distribution of EEG signals simultaneously in real-time. Some embodiments, for example, may advantageously be set up for EEG measurement within 5 minutes.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary Field EEG Monitoring System (FEMS) employed in an illustrative use-case scenario.

FIG. 2A, FIG. 2B, and FIG. 2C are schematic diagrams depicting an exemplary radio non-obstructing EEG acquisition system (RNOEAS).

FIG. 3 is a block diagram depicting an exemplary FEMS.

FIG. 4A and FIG. 4B are block diagrams depicting an exemplary brain health processing engine (BHPE) and brain health classification model (BHCM) in operation and configuration modes.

FIG. 5 is a flowchart illustrating an exemplary brain activity monitoring method using an exemplary RNOEAS.

FIG. 6A and FIG. 6B are schematic diagrams of an exemplary intersecting component of a RNOEAS.

FIG. 7A, FIG. 7B, and FIG. 7C are schematic diagrams of an exemplary multi-pin electrode.

FIG. 8A, FIG. 8B, and FIG. 8C are schematic diagrams of an exemplary electrode clip.

FIG. 9A and FIG. 9B are schematic diagrams of an exemplary central electrode.

FIG. 10A and FIG. 10B are schematic diagrams of an exemplary ear electrode.

FIG. 11A and FIG. 11B depict exemplary displays illustrating an exemplary configuration mode and an operation mode of an exemplary RNOEAS.

FIG. 12 is a flowchart illustrating an exemplary EEG signal monitoring method.

FIG. 13 is a flowchart illustrating an exemplary live brain activity visualization method.

FIG. 14 is a flowchart illustrating an exemplary abnormality identification method.

FIG. 15 depicts an exemplary method of training a classification model in a BHCM.

Like reference symbols in the various drawings indicate like elements.

### DETAILED DESCRIPTION OF ILLUSTRATIVE EMBODIMENTS

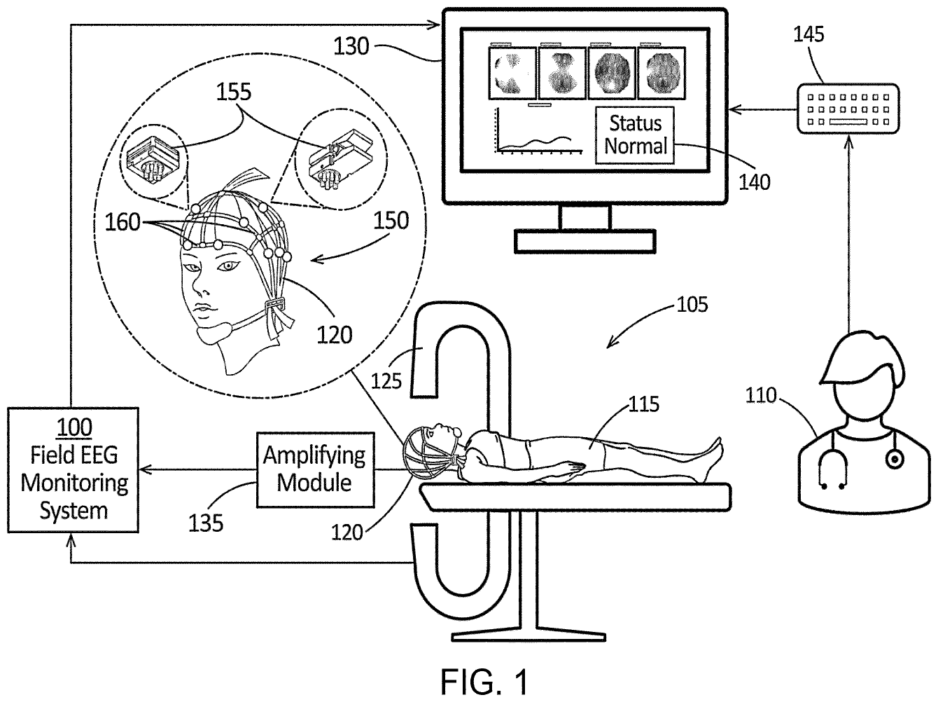

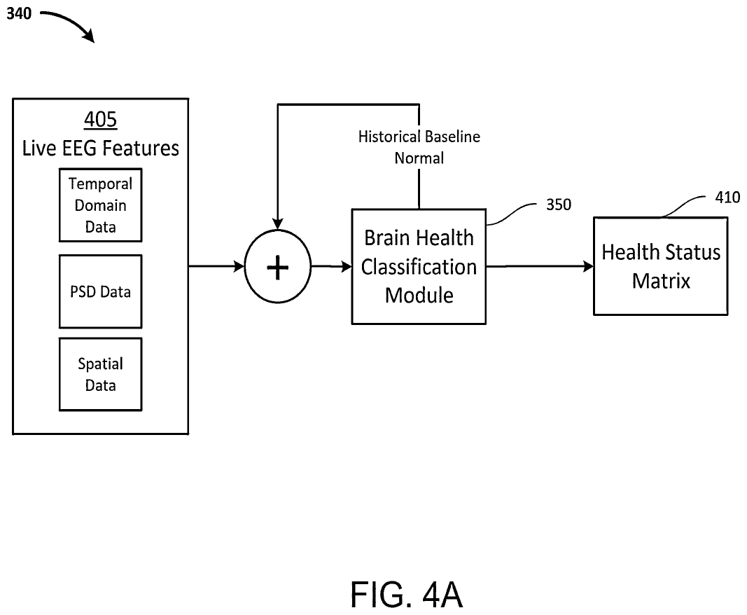

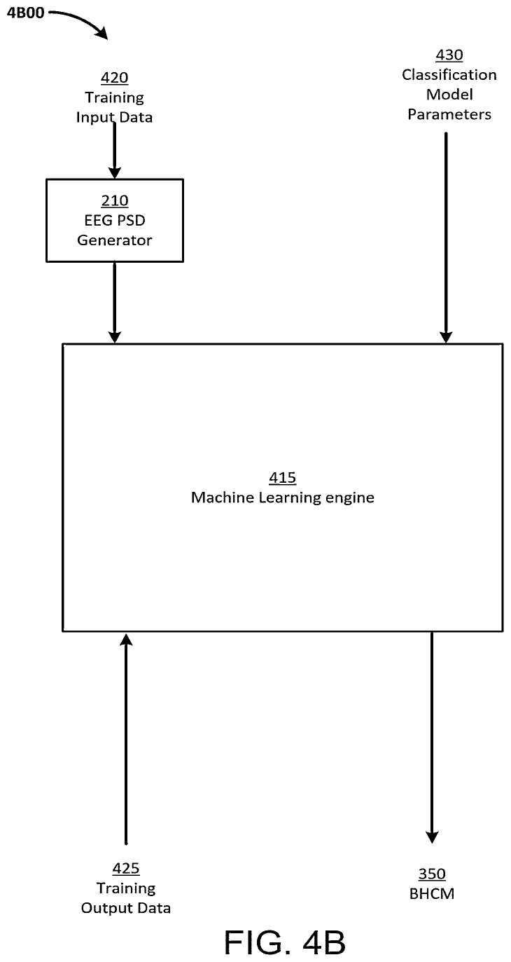

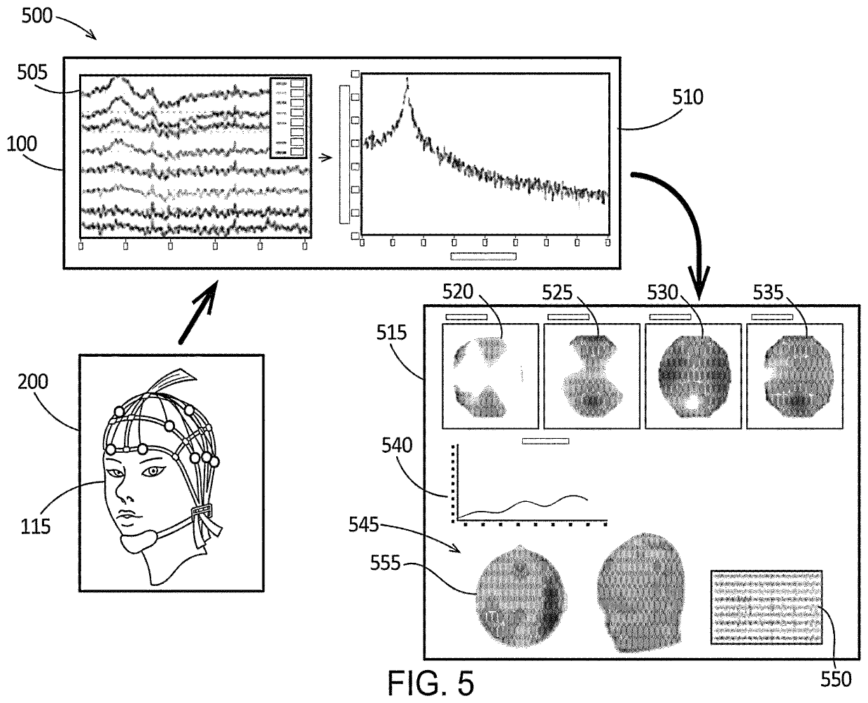

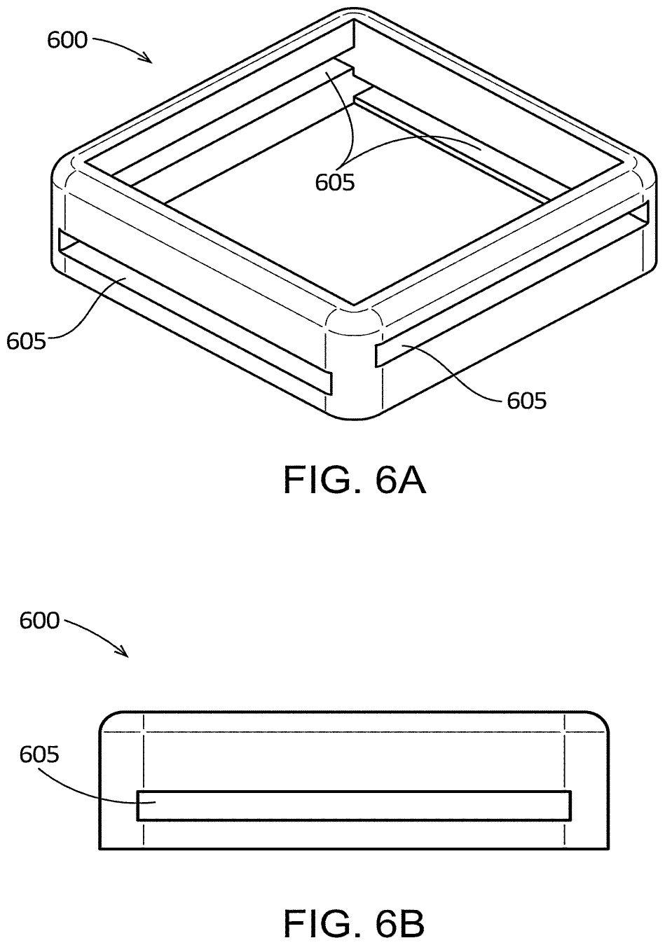

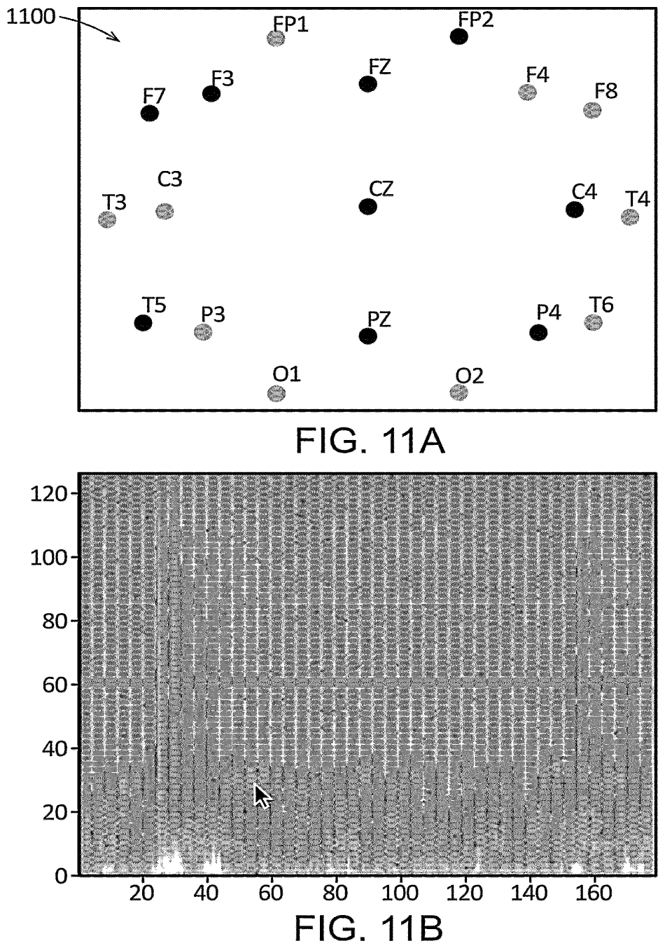

To aid understanding, this document is organized as follows. First, to help introduce discussion of various embodiments, a field EEG monitoring system (FEMS) is introduced with reference to FIGS. 1-3. Second, that introduction leads into a description with reference to FIGS. 4A-4B of some exemplary embodiments of a brain health processing engine. Third, with reference to FIG. 5, a data to visualization system is described in application to an exemplary FEMS. Fourth, with reference to FIGS. 6-10, the discussion turns to exemplary embodiments that illustrate exemplary radiotranslucent electrode units. Fifth, and with reference to FIGS. 11A-B, this document describes exemplary embodiments of display useful for configuration and operation of the FEMS. Sixth, this disclosure turns to a review of exemplary methods for various applications of the FEMS. Finally, the document discusses further embodiments, exemplary applications and aspects relating to the FEMS.

FIG. 1 depicts an exemplary Field EEG Monitoring System (FEMS 100) employed in an illustrative use-case scenario. For example, the FEMS 100 may be used in an operation theater 105. In this example, a surgeon 110 is performing an operation (e.g., an endarterectomy) with a patient 115 in the operation theater 105. For example, the operation may be a neurosurgery, a cardiac or vascular surgery, an open-heart surgery (e.g., using an Extracorporeal Membrane Oxygenation (ECMO) machine), an orthopedic surgery, and/or other surgery. In some examples, the operation may be a surgery in which the patient 115 is being operated on while under anesthesia.

In the depicted example, the patient 115 is monitored during the operation for various risks and/or complications that may be raised from the operation. For example, during a cardiac or vascular surgery, there may be a risk of brain embolism (e.g., endarterectomy where cutting clot out of carotid). For example, during an orthopedic surgery, the patient 115 may have a risk of clot formation. During neurosurgery, for example, brain activity may be monitored to determine response to surgery. In some examples, the surgeon 110 may have to constantly monitor the patient 115 against these risks.

The patient 115 is monitored, as an illustrative example shown in FIG. 1, by an EEG cap 120 and a fluoroscopy equipment 125. For example, the EEG cap 120 may include an EEG sensor array. For example, the EEG cap 120 may be an EEG headset. For example, the EEG cap 120 may be a dry EEG cap having dry leads/electrodes contacting the patient 115. For example, the EEG cap 120 and the fluoroscopy equipment 125 may be used to monitor brain activity and various health indicators of the patient 115 during the operation. For example, the FEMS 100 may use the EEG cap 120 to record brain waves at various parts of a head of the patient 115.

In some implementations, the EEG cap 120 using radiotranslucent dry electrodes may require a short setup time. For example, the EEG cap 120 with radiotranslucent dry electrodes may advantageously take less than 60 minutes (e.g., less than 45 minutes, 30 minutes, 15 minutes, 5 minutes) to be configured to be used for the patient 115. For example, a dry electrode may advantageously be operated without shaving a patient's body surface and/or without an intermediate signal conductor (e.g., gel).

Traditional EEG setup may, for example, require extensive time to setup properly to acquire signals useful for monitoring. The setup may, for example, require trained staff knowledgeable and experienced in the necessary conditions for obtaining a signal (e.g., surface preparation, gel type, gel quantity, electrode placement). As an illustrative example, in an emergency situation (e.g., when a patient is experiencing a stroke), trained staff may not be readily available. In an emergency situation, setup time required for traditional electrodes may, for example, directly impact whether a positive outcome (e.g., saving the patient's life and/or bodily functions such as brain tissue survival) may be achieved.

For example, the EEG cap 120 may advantageously enable staff with limited to no training in proper placement of EEG to quickly and easily set up EEG monitoring capable of capturing signals effective for monitoring. For example, when a patient is experiencing a stroke, time is of the essence. The EEG cap 120 may allow staff (trained, untrained) to quickly setup realtime EEG monitoring in an emergency situation.

By way of example and not limitation, some embodiments (e.g., such as described above) may advantageously enable a physician to obtain near-real time (e.g., intraoperatively) simultaneous EEG and radiography feedback on the progress of the operation. As an illustrative example, a neurosurgeon may advantageously visualize brain activity (e.g., by EEG) while also visualizing intracorporeal structures (e.g., tissue and/or tools, such as by radiography). For example, an operator (e.g., the neurosurgeon) may advantageously and simultaneously visualize physical objects (e.g., an obstruction, a tool location) by fluoroscopy while also monitoring the patient's brain activity response by EEG. Accordingly, some embodiments may advantageously solve a technical problem of acquiring near-real time monitoring of EEG activity without interrupting surgery and/or interrupting other monitoring modalities (e.g., radiography).

As an illustration, in the depicted example the surgeon may, for example, be performing an endovascular retrieval operation. For example, the surgeon may be operating to remove an embolus (e.g., a blood clot occluding blood flow to a portion of the body such as the brain). Successful removal of the embolus may, for example, lead to resumption of blood flow. The real-time monitoring (e.g., by EEG) may, for example, allow a surgeon to confirm successful removal by visualizing a change in brain activity (e.g., related to restoration of blood flow). For example, the EEG may advantageously confirm the surgeon's removal of the embolus as visualized (e.g., concurrently) by another imaging modality (e.g., fluoroscopy).

As an illustrative continuation of the example, during removal of the clot, smaller emboli may break off (e.g., smaller clots breaking off a larger clot during removal). One or more of the smaller emboli may flow downstream. A smaller embolus may, for example, obstruct blood flow at another location. For example, a smaller embolus may lodge in downstream vasculature, inducing additional stroke(s). The real-time electropotential monitoring may, for example, advantageously enable the surgeon to visually detect effects of the operation during the operation. For example, the surgeon may see (e.g., on a temporally and spectrally distributed visual display) that brain activity is weakening again in time. The surgeon may see (e.g., on a temporally dynamic display that is spatially and spectrally distributed display), a weakening in brain activity corresponding to the location of the new blockage(s). Accordingly, the surgeon may, for example, advantageously continue the operation to remove the blockage(s). For example, the surgeon may advantageously avoid ending the operation and bringing the patient out of anesthesia only to discover the existence, for example, of additional strokes induced during the operation. Accordingly, some implementations may advantageously reduce operation time, reduce total healthcare costs, reduce repeat operations, decrease morbidity and/or mortality, and/or improve patient recovery outcomes.

As an illustrative example without limitation, the EEG cap 120 and the fluoroscopy equipment 125 may, by way of example and not limitation, monitor a penumbra (e.g., related to a stroke) in the patient's brain (e.g., to determine whether the penumbra is becoming more or less active). In some implementations, the EEG cap 120 and the fluoroscopy equipment 125 may simultaneously monitor a brain activity of the patient 115. For example, the EEG cap 120 may be made of radio translucent materials to advantageously reduce interference to an image captured by the fluoroscopy equipment 125.

The operation theater 105 includes a display module 130 for displaying data received from the EEG cap 120 and the fluoroscopy equipment 125. For example, the displayed data may include live recordings and transmissions of information obtained from the EEG cap 120. In this example, the EEG cap 120 is operably coupled to the FEMS 100 via an amplifying module 135. For example, the amplifying module 135 may amplify raw data from the EEG cap 120 to be transmitted to the FEMS 100. In some implementations, the amplifying module 135 may be coupled to the FEMS 100 wirelessly. In some implementations, the amplifying module 135 may be coupled to the FEMS 100 via a wired connection. In some implementations, the amplifying module 135 may be coupled to the FEMS 100 with a combination of wired and wireless connections (e.g., through multiple layers of network construction, multiple computer devices).

In some implementations, the amplifying module 135 may be placed away from the patient 115. For example, the amplifying module 135 may include radio obstructing materials (e.g., metal). By placing the amplifying module 135 away from the patient 115, the amplifying module 135 may advantageously be prevented from obstructing a view of an image captured by the fluoroscopy equipment 125.

In some implementations, the surgeon 110 may use the display module 130 to obtain a near real-time feedback on various health indicators of the patient 115 during an operation. For example, upon receiving EEG data from the EEG cap 120 via the amplifying module 135, the FEMS 100 may analyze the EEG data to detect, for example, anomalies in the brain of the patient 115. For example, the FEMS 100 may provide an early detection of stroke of the patient 115 under anesthesia.

As shown, the display module 130 is currently displaying a live data from the EEG cap 120 and a status indication 140 of the patient 115. For example, a visual indicia may be generated as a function of the status indication 140. In some implementations, the FEMS 100 may process the live EEG data from the EEG cap 120 to generate a three-dimensional (3D) topography of the brain activity. For example, the FEMS 100 may combine image data received from the fluoroscopy equipment 125 to generate an overlay of the brain activity and the EEG signal. By combining live data from the fluoroscopy equipment 125 and the EEG cap 120, for example, the FEMS 100 may advantageously generate and display live topographical (e.g., 2D or 3D) visualization of EEG signals.

In some implementations, the FEMS 100 may process the live EEG data to generate one or more quantitative (qEEG) features. For example, the qEEG features may include features in a spatial domain, a frequency domain, a temporal domain, or a combination thereof. For example, the qEEG features may include power spectrum density (PSD) analysis. For example, the qEEG features may include absolute power of the EEG signals within one or more frequency bands. For example, the qEEG features may include relative power of the EEG signal within a frequency band relative to the total power of the EEG signal. For example, the qEEQ features may include spectral band ratios. For example, the qEEQ features may include a ratio of an alpha/beta ratio, theta/beta ratio, and/or delta/alpha ratio. For example, the qEEG features may include a coherence measures of synchronization between different EEG channels.

In some implementations, the FEMS 100 may perform spectrum analysis of the received digital signals from the EEG control unit 205. For example, the FEMS 100 may generate an output signal corresponding to a PSD data of the EEG signals.

In some implementations, the FEMS 100 may be configured to map spatial locations of each of the electrodes relative to the brain. For example, the EEG control unit 205 may generate, at a selected time, a data structure (e.g., a 2D or 3D data frame) of EEG signals corresponding to each spatial location of the brain.

As shown, the surgeon 110 may use a user input device 145 to control a visualization of the display module 130. For example, the surgeon 110 may select to expand one or more visualizations of the live EEG signals. In some implementations, the FEMS 100 may identify an area of concern (e.g., with high potential of health risk) and generate a visual indicium (e.g., using a different color of display, flashing, generating a warning sound) to notify the surgeon 110. For example, the surgeon 110 may use the user input device 145 to select the area and check on a zoom-in image of the area of concern. In some implementations, the surgeon 110 may use the user input device 145 to request the display module 130 to display a stream of historical development of a health concern. For example, the display module 130 may generate a (streaming) display of EEG signals for a particular area of the brain from a selective moment in time (e.g., 1 minute ago, 5 minutes ago, 10 minutes ago, since the operation started) to a present time. In some examples, the FEMS 100 may advantageously monitor the area of concern continuously to help the surgeon 110 to localize and/or verify removal of risk (e.g., blood clot in the brain).

As shown in a close-up view, the EEG cap 120 includes a supporting structure 150 and electrodes 155. For example, the electrodes 155 may releasably be coupled to the supporting structure 150. In some implementations, the supporting structure 150 may be adjustable to advantageously fit a size of the head/scalp of the patient 115. For example, the adjustability may advantageously improve comfort of the patient 115.

In some examples, with the adjustability of the EEG cap 120, medical professionals may customize a layout of the electrodes 155. For example, the customization may be performed to advantageously improve measurement results (e.g., by improving contractability of the electrodes 155 to the scalp).

In this example, the supporting structure 150 may include fabric bands 160. For example, the fabric bands 160 may include elastic nylon bands. In some implementations, the electrodes 155 may include various types of coupling modules that are configured to couple (e.g., clip on) on multiple places on the fabric bands 160. As an illustrative example, the surgeon 110 may customize positions of the electrodes 155 freely on the supporting structure 150. For example, when a surgery requires part of the head open for operation (e.g., for a neurosurgery), the surgeon 110 may arrange the supporting structure 150 and the electrodes 155 in a way without obstructing the operation.

In some implementations, the supporting structure 150 and the electrodes 155 may include radiotranslucent materials. For example, the radiotranslucent material may allow radiation beams (e.g., X-ray) of the fluoroscopy equipment 125 to pass through without distorting an image captured by the fluoroscopy equipment 125. For example, the fluoroscopy equipment 125 may include a computational tomography (CT) scanning and/or X-rays. The minimally radio-obstructing EEG cap 120 may advantageously allow a combination of both the radiographic images and the EEG signals to determine an exact location of abnormal brain activity, for example.

As shown in this example, the status indication 140 is displaying a normal status. For example, the status indication 140 may include a visualization graphic representing a status of, for example, normal, stroke, and abnormal but without stroke. In some implementations, the FEMS 100 may generate a real-time inference based on the received EEG signals from the EEG cap 120. For example, the FEMS 100 may generate an indication of abnormality as a function of the qEEG features based on the EEG signals.

In some implementations, the display module 130 may display a visualization of the qEEG features. For example, the qEEG features may be updated in real-time or near real-time (e.g., every second, every 5 seconds, every 10 seconds). For example, the visualization may include a heatmap indicating a probability of abnormality in various locations of the brain. For example, the heatmap may be a three-dimensional graphic generated by the FEMS 100.

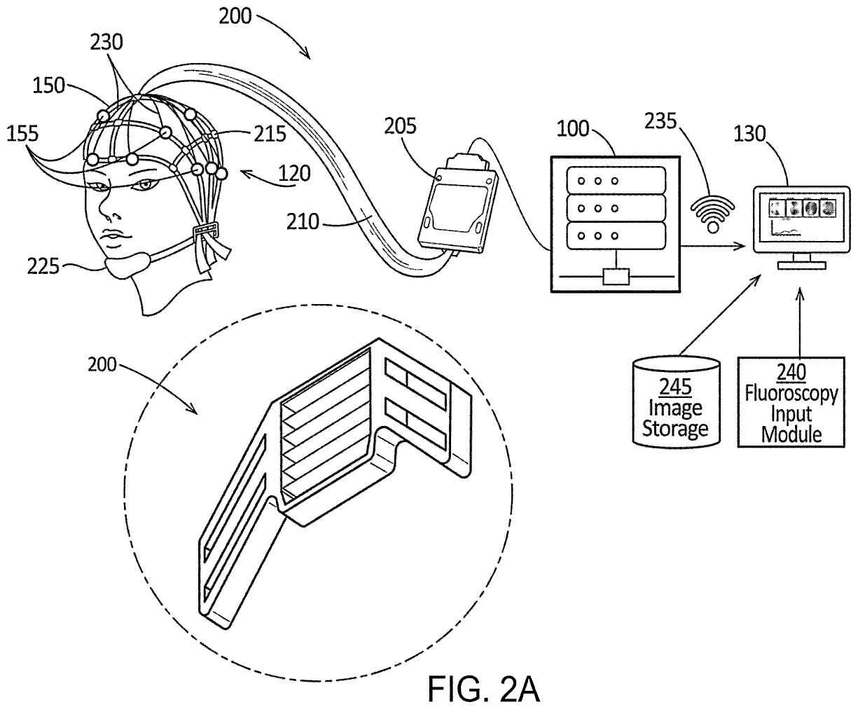

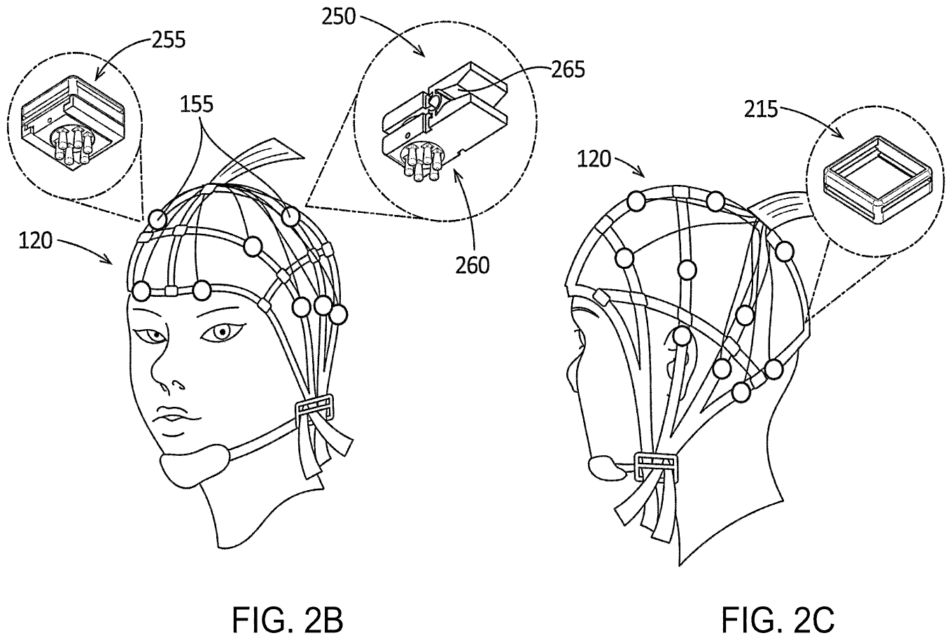

FIG. 2A, FIG. 2B, and FIG. 2C are schematic diagrams depicting an exemplary radio non-obstructing EEG acquisition system (RNOEAS 200). For example, the RNOEAS 200 may be used in the operation theater 105 as described in FIG. 1. As shown in FIG. 2A, the RNOEAS 200 includes the EEG cap 120 and an EEG control unit 205. For example, the EEG control unit 205 may include the amplifying module 135. As shown, the EEG control unit 205 may be connected to the EEG cap 120 via a connection bus 210.

In various implementations, the EEG control unit 205 may be relocatable. For example, the EEG control unit 205 may be flexibly placed at a position where the patient 115 may be allowed to lay down. For example, the EEG control unit 205 may be placed near a shoulder of the patient 115. In some examples, the EEG control unit 205 may be flexibly placed near the patient 115 on, for example, a side rack near the patient 115. For example, the EEG control unit 205 may advantageously allow the patient 115 to be repositioned during the operation without disconnecting the EEG control unit 205 nor the EEG cap 120.

The connection bus 210, as depicted, is coupled to the EEG control unit 205 located away from the EEG cap 120. For example, when the EEG control unit 205 is remotely connected, the EEG control unit 205 may advantageously prevent the EEG control unit 205 from obstructing a view of any radio imaging device in an operation concurrently used with the RNOEAS 200.

In the depicted example, the electrodes 155 are coupled to the supporting structure 150. In some implementations, the electrodes 155 may be placed anywhere on the electrodes 155 to advantageously improve variability of the EEG cap 120. As shown, the supporting structure 150 includes a lattice structure. For example, the lattice structures may be generated using intersecting modules 215. For example, the intersecting modules 215 may be 3D printed. In some examples, the intersecting modules 215 may include apertures that allow straps of the fabric bands of the supporting structure 150 to overlap perpendicularly. Some embodiments of the intersecting modules 215 are described with reference to FIGS. 6A-B.

As shown, the bands (e.g., the fabric bands 160) of the supporting structure 150 are threaded through a ladder lock slider 220. For example, a user may tighten certain areas of the EEG cap 120 using the ladder lock slider 220 for a better fit at a head of the patient 115. For example, the tightening of the EEG cap 120 may be performed by pulling ends of the bands from the head. In this example, the EEG cap 120 also includes a chin strap 225. For example, the chin strap 225 may hold the EEG cap 120 onto the patient 115 to advantageously maintain signal contact from the scalp to the electrodes 155. This chin strap 225, in some implementations, may include a nylon band and a cushioned chin cup. For example, the nylon band and the cushioned chin cup may advantageously ensure comforts of the patient 115.

Leads 230 are coupled from the electrodes 155 to and/or bundled into the connection bus 210. In some implementations, electrical activities may be collected from the electrodes 155 and passed through the leads 230 to the EEG control unit 205.

In some implementations, the leads 230 may include a 42 AWG 150V black 100 wire. For example, the leads 230 may be insulated by fluorinated ethylene-propylene (FEP). In some implementations, an insulating material of the leads 230 may include a radiotranslucent polymer. In some examples, metallic materials like copper tend to appear in radiographic images (e.g., X-ray/CT images) For example, a X-ray attenuation coefficient of pure copper may be sufficiently low compared to a dense concentration of calcium (e.g., the skull bone). Accordingly, an unalloyed bare copper wire may be effectively radiotransparent. For example, the copper wire may include a small diameter (e.g., less than 0.07 mm, less than 0.1 mm). For example, a 42 AWG copper wire may minimize an amount of detection during imaging. For example, the EEG cap 120 may be effectively radiotranslucent when, comparing to a bone in a radiographic image, the maximum opacity of the EEG cap 120 is less a threshold (e.g., 80%, 70%, 50%, 30%) of the bone. In some implementations, to further reduce imaging artifacts, the leads 230 may be attached to the electrodes 155 by using Ag/Cl paint as an adhesive.

In some implementations, the EEG control unit 205 may amplify the received signal to the FEMS 100 data. For example, the FEMS 100 may receive the amplified signal (e.g., via USB-C connection). For example, the FEMS 100 may generate visualization of the received signal (e.g., based on qEEQ features generated) and display on the display module 130. In some implementations, the visualization may be livestreamed on a local network 235. For example, the local network may be a wired network. In this example, the local network 235 is a Wi-Fi network.

In some implementations, the EEG control unit 205 may include an analog to digital converter (ADC) and one or more communication modules (e.g., a wireless communication module, a wired communication port). For example, the EEG control unit 205 may receive analog EEG signals from the connection bus 210 and convert the analog EEG signals into digital signals.

In various implementations, the EEG cap 120 may include dry electrodes with at least partially radio translucent properties to reduce interference with the fluoroscopy equipment 125. In some implementations, the EEG cap 120 may include wet electrodes modified to fit in a cap form. In some examples, the dry electrodes or the modified wet electrodes may advantageously allow the EEG cap 120 to be easily applied to the patient 115.

The display module 130, for example, may generate a display as a function of qEEG features received from the FEMS 100. In some examples, the display module 130 may include a general purpose computing device. In some implementations, the display module 130 may also receive inputs from a fluoroscopy input module 240. For example, the fluoroscopy input module 240 may transmit live data received from the fluoroscopy equipment 125 and image storage 245. In some implementations, the image storage 245 may include various images related to the patient 115 and the operation. For example, the image storage 245 may include computed tomography (CT) images, angiogram images, and/or Magnetic resonance imaging (MRI) images of the patient before the operation. In some implementations, the display module 130 may retrieve one or more of the stored images from the imaging module.

In some implementations, the display module 130 may dynamically generate various sets of images to be displayed. For example, the display module 130 may generate a live topographical display (e.g., in 2D, 3D) of PSD-based visualization of EEG signal. For example, the live topographical display may advantageously visualize brain activities (e.g., ischemia, hemorrhage, seizure) in real time. In some examples, the Display module 130 may generate a live visual comparison (e.g., by overlaying more than one image) of actual EEG with previous or intermittent imaging (e.g., from the fluoroscopy input module 240 or the image storage 245).

In some implementations, by overlaying the generated PSD-based visualization of the EEG signal with images stored in the image storage 245, the display module 130 may advantageously generate a spatially overlay of the brain activity with a fixed image target from the patient 115 before the operation.

In some implementations, the EEG cap 120 may include 19 recording channels and 2 reference and ground electrodes. For example, the display module 130 may include a display engine that continually displays an impedance map and voltage data for all channels.

In an illustrative example, using the EEG cap 120 with radio translucent leads, the display module 130 may dynamically generate a real-time display by processing images received from the FEMS 100, the fluoroscopy input module 240, and/or the image storage 245. In some examples, the FEMS 100 may advantageously allow the surgeon 110 to continuously monitor and track risk areas of the patient 115 during a surgery operation.

As shown in FIGS. 2B and 2C, a front perspective view and a back perspective view of the EEG cap 120 are shown. In this example, the EEG cap 120 may include elastic bands (e.g., in nylon) organized in a lattice structure. For example, the elastic bands may advantageously fit heads with a range of circumference (e.g., between 50-75 cm). For example, with the bands running along the head, the EEG cap 120 may allow a continuous freedom in placing electrodes around the head of the patient 115. For example, the fabric bands 160 may be organized in a lattice format to advantageously expose a scalp for easy access and prevent sweating.

As shown in FIG. 2A, the EEG cap 120 includes a clip-on electrode module 250 and a threaded through electrode module 255. The clip-on electrode module 250 and the threaded through electrode module 255 may each include a threaded through electrode unit 260. For example, the electrode unit 260 may be releasable coupled to the clip-on electrode module 250 and/or the electrode module 255. For example, the threaded through electrode unit 260 may include one or more 3D-printed electrodes. For example, the threaded through electrode unit 260 may include 4 electrodes. For example, the electrode unit 260 may include 6 electrodes.

For example, the electrode unit 260 may include dry electrodes. In some implementations, the electrode unit 260 may include a radiotranslucent body coated with a conductive layer (e.g., a conductive ink). For example, the conductive layer may include silver/silver chloride (Ag/AgCl) ink. As shown, the clip-on electrode module 250 and the threaded through electrode module 255 may be releasably coupled to the nylon bands of the EEG cap 120. In some implementations, the electrode unit 260 may include spring loaded pins to advantageously maintain a firm contact between the electrode unit 260 and a scalp of the patient 115.

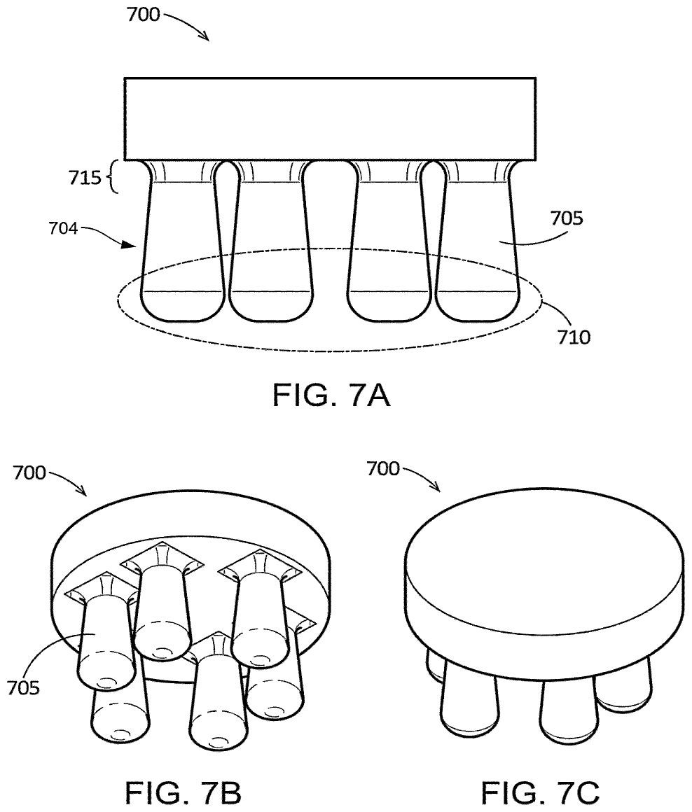

In some implementations, the electrode unit 260 having radiotranslucent polymeric electrode body with a conductive coating (e.g., silver chloride) applied to a polymeric body may advantageously be visually translucent under a radiograph. For example, the electrode body may be non-conducting. In other examples, the body of the electrode unit 260 may include other non-metallic bodies. For example, the non-metallic body may be less than a density threshold (e.g., less than 1.5 g/cm³, less than 0.5 g/cm³) to be effectively radiographically translucent. Various embodiments of the electrode unit 260 are further described with reference to FIGS. 7A-C.

In this example, the clip-on electrode module 250 may include an alligator clip 265 (e.g., 3D printed). For example, the alligator clip 265 may clamp onto the nylon bands of the EEG cap 120. For example, the clip-on electrode module 250 may be clipped or unclipped from the EEG cap 120. For example, the releasably coupling mechanism may advantageously make the EEG cap 120 customizable.

In some implementations, the electrode module 255 may be placed at central positions of a scalp (e.g., on a midline frontal electrode position (Fz), on a midline central electrode position (Cz), on a midline parietal electrode position (Pz)). For example, at these positions, the clip-on electrode module 250 may be insufficient to accurately place the electrodes 155 at a required location. For example, the electrode module 255 may be coupled to the EEG cap 120 using a sliding mechanism.

In various implementations, the EEG cap 120 may include substantially minimum metal to advantageously enhance radio transparency. For example, the EEG cap 120 may include zero metal except from the silver particles inside the conductive ink. In some implementations, the EEG cap 120 may transmit EEG signal from the electrodes 155 (e.g., the clip-on electrode module 250 and the threaded through electrode module 255) through a small wire (e.g., the leads 230). For example, the small wire may be a copper wire. For example, the copper wire may include a diameter of less than 100 American Wire Gauge (AWG) (e.g., 30 AWG, 42 AWG, 56 AWG, 80AWG). For example, the smaller diameter copper wire may effectively be radiotranslucent. For example, the copper wire may barely be seen in radiographic images.

In some implementations, ends of the small wires may be soldered to the electrocardiogram (EKG) lead wires (e.g., of the EEG control unit 205). For example, a male end of the EKG lead wire may be plugged into the female end of a touchproof adaptor to ensure a continuous connection. Soldered connections appear during imaging, for example, may be removed from a concern because the EKG lead wires and the EEG control unit 205 may be flexibly placed away from a body for radiography.

In various implementations, the EEG cap 120 may include radiotranslucent features to advantageously reduce severity of imaging artifacts in CT and X-ray imaging modalities, for example, caused by metallic electrodes. Being customizable, the EEG cap 120 may advantageously simplify a setup process of the EEG cap 120 in configuring a successful contact to the scalp.

As shown in FIG. 2C, a closed-up view of the intersecting modules 215 is shown. For example, the intersecting modules 215 may enable the EEG cap 120 to be resized for different sized and/or shape of heads. For example, without rigid attachments between the supporting structure 150, when the supporting structure 150 is placed on a head of the patient 115, the supporting structure 150 may be moved around to customize placements of the electrodes 155. Some embodiments of the intersecting modules 215 are further described with reference to FIGS. 6A-B.

In various implementations, an EEG cap (e.g., the EEG cap 120) may include a support structure (e.g., the supporting structure 150) adjustably fits onto a scalp of a patient. For example, the EEG cap 120 may include a communication system (e.g., the EEG control unit 205) that includes a network of wires (e.g., the leads 230). For example, the EEG cap 120 may include a clip-on electrode unit (e.g., the clip-on electrode module 250) coupled to the EEG control unit 205 through the leads 230. For example, the clip-on electrode module may include a spring-loaded recording channel (e.g., the electrode unit 260) for dry electrodes. For example, the spring-loaded recording channels may be configured to conduct EEG signals from the scalp to the communication system. For example, the soft support may be radio translucent and the clip-on electrode unit may be radio translucent except a visually non-obstructing wire at each spring-loaded dry electrode. For example, brain waves of the patients are continuously recorded without obstructing a view of a radioactivity imaging tool.

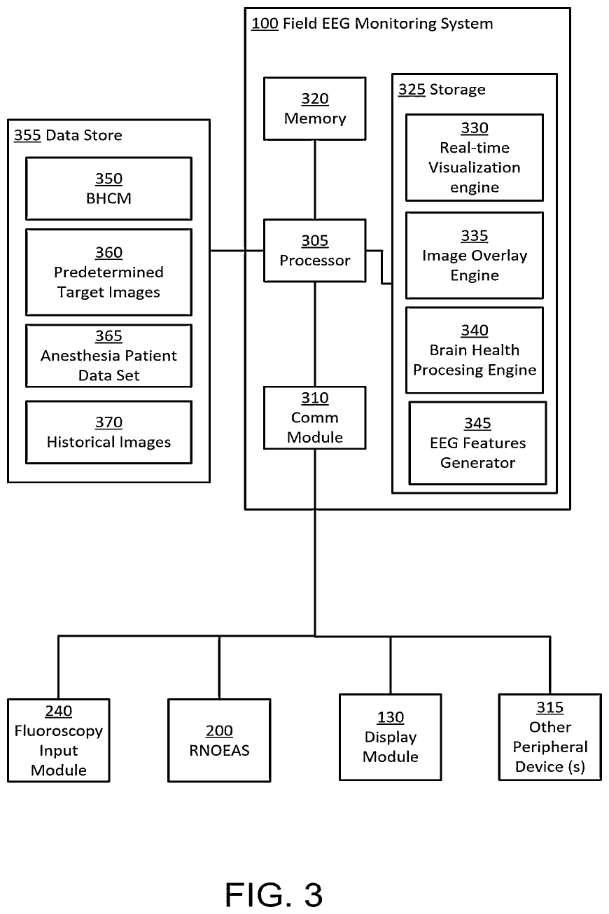

FIG. 3 is a block diagram depicting an exemplary FEMS (e.g., the FEMS 100). The processor 305 may, for example, include one or more processing units. The processor 305 is operably coupled to a communication module 310. The communication module 310 may, for example, include wired communication. The communication module 310 may, for example, include wireless communication. In the depicted example, the communication module 310 is operably coupled to the fluoroscopy input module 240, the RNOEAS 200, and the display module 130. For example, the FEMS 100 may receive input from the fluoroscopy input module 240 and the RNOEAS 200 to generate a visualization output at the display module 130.

the FEMS 100, and other peripheral devices 315. For example, the peripheral devices 315 may include user input devices (e.g., a keyboard, a user interface pointer device). For example, the other peripheral devices 315 may include external data storage devices of, for example, training data. For example, the other peripheral devices 315 may include an external data storage device of electronic health records of patients (e.g., the patient 115). For example, the other peripheral devices 315 may include the image storage 245 as discussed with reference to FIG. 2A.

The processor 305 is operably coupled to a memory module 320. The memory module 320 may, for example, include one or more memory modules (e.g., random-access memory (RAM)). The processor 305 includes a storage module 325. The storage module 325 may, for example, include one or more storage modules (e.g., non-volatile memory). In the depicted example, the storage module 325 includes a real-time visualization engine (RVE 330), an image overlay engine (IOE 335), a penumbra identification engine (BHPE 340), and an EEG features generator (EFG 345). The RVE 330 may, for example, generate images for display based on received PSD data from the EFG 345. In some implementations, the generated images may be 2D. In some implementations, the generated images may include both 2D images and 3D images. For example, a user may use a control device (e.g., the user input device 145) connected to the display module 130 via the communication module 310 to navigate various images generated by the RVE 330. For example, the surgeon 110 may use the RVE 330 to generate a video stream of a time-domain development of EEG signals. For example, a user may rotate the image and zoom in to various cross-sections to monitor various areas of the brain.

The IOE 335, in some implementations, may overlay one or more images from different sources to generate a display for the display module 130. In some examples, the IOE 335 may display images received from the fluoroscopy input module 240 and the RVE 330 on the display module 130 side by side. In some examples, the IOE 335 may overlay the images received from the image storage 245 and the RVE 330 on the display module 130 to advantageously identify a difference between a real time image and one or more of the stored images. In some examples, the IOE 335 may overlay live images received from the fluoroscopy input module 240 and the RVE 330, and historically (or previously generated) images on the display module 130 to advantageously identify improvement or deterioration of risk areas of the patient 115.

In some implementations, the BHPE 340 may apply a brain health classification model (BHCM 350) to identify abnormal areas of the patient 115 by processing EEG (e.g., qEEG features) generated by the EFG 345. For example, the 345 may generate spatial features, temporal features, and/or power density spectrum features using EEG signals received from the RNOEAS 200. In some implementations, the BHPE 340 may generate PSD data of EEG signals received from the RNOEAS 200 to generate side by side, for example, an alpha range, a beta range, a delta range, and a theta range PSD of the EEG signals using the RVE 330.

In some implementations, the EFG 345 may also apply images generated by the IOE 335 to the BHCM 350 to classify health status of (e.g., various areas of) a brain of the patient 115. For example, the BHCM 350 may be artificially intelligently trained to determine penumbra areas from multiple EEG PSD visualized images. For example, the IOE 335 may generate simultaneously (e.g., a side-by-side and/or an overlayed image) of the EEG features generated by the EFG 345 and images from the fluoroscopy input module 240 at the display module 130. Some embodiments of the BHPE 340 are described with reference to FIGS. 4A-B. The BHPE 340 may, for example, determine whether progress is being made based on temporally distributed images (e.g., determine positive progress if the penumbra is becoming more active).

In some implementations, the BHCM 350 may include an ensemble of decision trees. For example, the ensemble of decision trees may minimize a likelihood of overfitting the BHCM 350 a cohort of patients in a training set (as described with reference to FIG. 4B). Additionally, the ensemble of decision trees may introduce a diverse set of features into the BHCM 350. For example, the features may provide a robust system for detecting large vessel occlusion. For example, the features may also be advantageously detect abnormal signals related to underlying pathology of the patient 115.

The processor 305 is further operably coupled to the data store 355. The data store 355, as depicted, includes the BHCM 350, predetermined target images 360, an anesthesia patient data set 365, and historical images 370. For example, the IOE 335 may generate overlaying images by overlaying a live image data with the predetermined target images 360. For example, the predetermined target images 360 may include CT images, MRI images, and/or other radiological images produced for the patient 115 before the operation. The BHCM 350 may, for example, be retrieved (e.g., by the BHPE 340) to identify an abnormality and/or target (e.g., penumbra areas in the brain). The historical images 370, in some examples, may include previous images from a live image data feed from the current operation. In some implementations, the RVE 330 may use the historical images 370 to identify improvement or deterioration of risk areas of the patient 115.

In various implementations, a display system (e.g., the FEMS 100) may include a radio translucent electrode cap (e.g., the EEG cap 120, the RNOEAS 200) configured to detect EEG signals from a body part the patient. For example, the electrode cap may be radiotranslucent. For example, a concurrent view of radiographic images of a substantially same body part may substantially be unobstructed by the electrode cap.

For example, the display system may include a computer system coupled to the radiographic input and the electrode cap to receive the image signals and the EEG signals. For example, the computer system may include a brain health classification model (e.g., the BHCM 350) to classify a brain health using temporally, spectrally, and spatially distributed EEG features (e.g., generated by the EFG 345). For example, the display system may, based on a classification result from the BHCM, generate a simultaneous display of a real-time distribution of EEG features. For example, a detection of abnormality in brain health may be performed before the anesthetic patient regains consciousness. In some examples, a dynamic display of the real-time distribution of EEG features may be generated within 30 minutes. In some examples, a dynamic display of the real-time distribution of EEG features may be generated within 60 minutes. In some examples, a dynamic display of the real-time distribution of EEG features may be generated within 120 minutes. In some examples, a dynamic display of the real-time distribution of EEG features may be generated within 180 minutes.

In some implementations, the FEMS 100 and the RNOEAS 200 may be deployed quickly in an emergency situation. As an illustrative example without limitation, a patient may be suffering from an emergency stroke. For example, the patient might need to be cared for with a diagnosis of brain health in such an emergency situation. Using a non-adjustable radiographically obstructing EEG cap or with wet electrodes, medical personnel (e.g., an emergency medical technician (EMT), an emergency room doctor) may need at least 45 minutes to set up an EEG measuring tool. Additionally, the diagnosis might take hours to be interpreted by technical personnel (e.g., a radiologist) before a treatment plan is to be devised and executed. Moreover, when other radiographic images (e.g., X-ray images, a CT-Scan) are needed for further diagnosis, the EEG cap may need to be removed because radiographic images would be obstructed by the cap. As a result, for example, the patient might lose critical time with the best chance of recovery. The RNOEAS 200, for example, may solve a technical problem with a technical solution by using an adjustable supporting structure 150 (e.g., using the ladder lock slider 220 and the chin strap 225), a radiotranslucent electrodes and body (e.g., the electrode module 255), and highly repositionable electrode units (e.g., using the clip-on electrode module 250), and a quick EEG features analysis and display system (e.g., the FEMS 100). In various embodiments, the FEMS 100 and the RNOEAS 200 may advantageously reduce a total diagnosis time in critical situations like during an emergency stroke.

FIG. 4A and FIG. 4B are block diagrams depicting an exemplary brain health processing engine (BHPE) and an exemplary brain health classification model (BHCM) in operation and configuration modes. As shown in FIG. 4A, the BHPE 340 includes the BHCM 350, In this example, the 340 may use the BHCM 350 to determine, over time, an improvement, or a deterioration of brain health of the patient 115. In this example, the BHPE 340 receives Live EEG Features 405 as input. For example, the Live EEG Features 405 may be generated by the EFG 345. As shown, the Live EEG Features 405 includes time domain data, PSD data, and spatial data.

In some implementations, the Live EEG Features 405 may include representation from the temporal, spectral, and spatial domains. For example, the temporal features include the sample entropy and Hurst exponent computed on the signal captured from each electrode. Some of the Live EEG Features 405 may be generated from the spectral domain, in some implementations. For example, the Live EEG Features 405 may include a Relative power across the frequency bands of interest (e.g., Delta (0-2 Hz), Theta (3-5 Hz), Alpha (6-11 Hz), Low-beta (12-18 Hz), High-beta (19-29 Hz)). For example, the Live EEG Features 405 may include a sum total power for each frequency band. For example, the Live EEG Features 405 may include a relative alpha band power minus sum of power in beta band frequencies. For example, the Live EEG Features 405 may include a relative delta band power minus sum of power in theta frequency range. For example, the Live EEG Features 405 may include a relative delta band power divided by alpha power (e.g., a “Delta-Alpha ratio”). For example, the Live EEG Features 405 may include a difference in alpha and beta power divided by difference in delta and theta powers (e.g., a “Delta-Theta Alpha-Beta Ratio”). For example, the Live EEG Features 405 may include an Alpha and Theta coefficients. For example, the Live EEG Features 405 may include a Theta to Alpha Transition frequency.

In some implementations, some spatial features may be used to analyze changes in power across the span of electrodes placed on the scalp. For example, an unbalanced power during a substantial period of time (e.g., where one electrode expresses higher power than its laterally corresponding electrode) may indicate a decrease in the brain's electrical activity at the location of the electrode lacking in power output. For example, by computing the Inter-Hemispheric Amplitude Ratio (IHAR) for each electrode pair (i.e. FP1-FP2) and/or the Brain Symmetry Index (BSI), the BHPE 340 may generate an averaged difference in spectral power between the two hemispheres of the brain. For example, the BHPE 340 may generate and/or use the BHCM (e.g., a series decision trees) for each feature set to devise models for the temporal, spectral, and spatial domains. For example, these models may be aggregated into a stacked ensemble. In some embodiments, the BHCM 350 may include a multiclass classification. For example, the BHPE 340 may predict large vessel occlusion, other smaller strokes, and/or other neurological abnormalities that are common in the prehospital setting.

In this example, the BHPE 340 compares the Live EEG Features 405 with a historical baseline (e.g., normal conditions, previous conditions). In some implementations, the BHPE 340 may generate a health status matrix 410 as a function of the historical baseline normal and the Live EEG Features 405. For example, the health status matrix 410 may include a normal or an abnormal signal at various locations of a brain (e.g., based on the recording channels). For example, using a live classification model, the BHPE 340 may identify stroke or ischemia from the received (live) EEG Features 405. In some implementations, the RVE 330 may generate a display at the display module 130 based on the health status matrix 410.

In some examples, the BHPE 340 may use the BHCM 350 to determine whether there is a positive or a negative trend at the patient 115. For example, the surgeon 110 may be operating to restore normal blood flow to a brain of the patient 115. The surgeon 110 may have identified a penumbra area associated with brain tissue that is affected by a loss of blood flow (e.g., embolism-induced) but may have a potential to be rescued. By signaling a positive or negative trend (e.g., is the penumbra region showing increasing brain activity), the surgeon 110 may receive additional guidance on whether changes in the remedies are required, for example.

In some implementations, the BHPE 340 may further include additional inputs including clinical ratings and/or patient data. For example, the additional input may be received from an external data store. For example, the additional inputs may improve classification accuracy, improve precision, and/or reduce recalls of classification results.

As shown in FIG. 4B, an exemplary machine learning engine is shown for stroke prediction. In this example, a machine learning engine 415 includes a machine learning model. The machine learning model may, by way of example and not limitation, include a DRF, a DNN, a GBM, and other supervised classification model.

A set of training data is applied to the machine learning engine 415 to train the machine learning model. For example, the set of training data may include the anesthesia patient data set 365. The training data includes a set of training input data 420 and a set of training output data 425. The set of training input data 420 may include historical EEG signal recordings and stroke classifications. The training input data 420 may include, for example, 1-D input vectors generated from a databank of the anesthesia patient data set 365. In some examples, the set of training input data 420 may include data input of patients in general.

The set of training output data 425 may include, corresponding to each of the input vectors, positive and negative labels determined for stroke identification. As an illustrative example, the training input data may be generated based on a k-fold cross validation parameter. Various implementations for identifying stroke using a classification model are described in PCT Patent Application Serial No. PCT/US2023/060120, titled “Stroke Prediction Multi-Architecture Stacked Ensemble Supermodel,” filed by Ezekiel Fink, et. al., on Jan. 4, 2023. Specifically, various systems and methods for a filed stroke classification system are described with reference to FIGS. 1B-C, FIG. 3, and FIGS. 4-6, and [0036-46] and [0057-77]. This application incorporates the entire contents of the foregoing application(s) herein by reference.

During operation, classification models parameters 430 may be provided as inputs to the machine learning engine 415. For example, the classification models parameters 430 may include a type of the classification model, parameters of the classification model (e.g., depth, size, input size, output size). As shown, the machine learning engine 415 generates the BHCM 350 based on the classification models parameters 430.

In an illustrative example, the BHCM 350 may include the models selected based on a series of 3-fold cross validated grid searches for each class of feature types (e.g., PSD features, qEEG features, time domain features, and spatial domain features) and a 3-fold cross validated grid search on the entire feature set (e.g., all features) based on the classification models parameters 430. For each category of features, in some implementations, a series of model types may be developed. For example, the time domain features models may include a 3-folds cross-validated grid search collection of distributed random forests, gradient boosted machines and feed forward neural networks. The hyper-parameter grid for the random forest may, for example, search an optimal combination of number of trees (e.g., {200, 300, 350}). For example, a maximum depth of trees may be {20,21,22,23,24,25,26,30}. The classification models parameters 430 of an optimal gradient boosted machines may include a number of trees {200, 300, 368, 500}, a maximum depth of trees {10, 15, 20, 25, 30} and a learning rate {0.05, 0.07, 0.1, 0.2, 0.3}. The feed forward neural network's classification models parameters 430 may define the model architectures. For example, the number of nodes per hidden layer may be {[30,30,30,30,30], [30,30,30], [25,20,15], [15,10,8], and [12,12,12]}. The activation functions attempted may be set by the machine learning engine 415 as {hyperbolic tangent, rectified linear unit, rectified linear unit with dropout}. The number of maximum training epochs may be specified as [90, 105, 120, and 200], for example.

The PSD feature models may be created with a similar set of models as with the time series features and consisted of a 3-folds cross-validated grid search collection of distributed random forests, gradient boosted machines and feed forward neural networks. The random forest, for example, may include classification models parameters 430 may include grid parameters grid searched. For example, this may include a number of trees {200, 256, 300, 325, 360} and maximum depth of trees {20, 25, 30, 35}. The gradient boosted machine's grid parameters grid searched may include a number of trees {200, 300, 368, 500}, a maximum depth of trees {7, 10, 15, 20, 25, 30}, and a learning rate {0.009, 0.05, 0.07, 0.1, 0.2, 0.3}.

As an illustrative example, the qEEG features models may be built and assessed by 3-folds cross-validated grid search using a set of distributed random forests with predetermined classification models parameters 430. For example, the electrode module 255 may include a number of trees {125, 200, 250, 300} and a maximum tree depth {10, 15, 20, 25, 30}. For example, the all features model may be generated by 3-folds cross-validated grid search using a set of XGBoost models. For example, the classification models parameters 430 of the XGBoost models may include a number of trees {100, 125, 150, 250, 300, 368, 400} and a maximum depth {10, 15, 20, 25, 30, 35}.

In some implementations, the machine learning engine 415 may generate SHAP (Shapley Additive exPlanitions) values of the test data to, for example, be calculated to provide a level of understanding and explainability to brain health classification. Various embodiments may be configured to provide a clinician evaluating a brain health with explainable predictive analytics.

FIG. 5 depicts an exemplary spatially and bandwidth distributed EEG display system (SBDEDS 500) using an exemplary FEMS and an exemplary RNOEAS. In this example, the RNOEAS 200 transmits raw data 505 from the patient 115 to the FEMS 100. For example, the RNOEAS 200 may transmit received EEG signals wirelessly to the FEMS 100. In some implementations, the EEG control unit 205 may push data through an outlet to a (wireless) local network to the FEMS 100. For example, the raw data 505 may include an impedance data stream. In some implementations the raw data 505 may include a voltage data stream. In some implementations, the surgeon 110 may selectively view and/or record the impedance and the voltage data stream using the user input device 145.

As shown, the raw data 505 are processed into various EEG features 510. For example, the FEMS 100 may filter and remove noise from the raw data 505 to generate the EEG features 510. For example, the EEG features 510 may include spatially distributed features, temporal distributed features, and PSD features. The FEMS 100 may, for example, transmit the various EEG features 510 to be displayed (e.g., at the display module 130). In this example, the display module 130 displays the various EEG features 510 simultaneously in a display 515. For example, the display 515 may include a spatially distributed heatmap 520 of a brain. For example, the display 515 may include a beta band heatmap 525 of the brain. For example, the display 515 may include a delta band heatmap 530 of the brain. For example, the display 515 may include a theta band heatmap 535 of the brain. For example, the display 515 may include a time-domain health level 540 of the brain.

In this example, the display 515 also includes a simultaneous real time display (SRTD 545). For example, the SRTD 545 may include a real-time multi-channel EEG signal display 550 (received from the RNOEAS 200) and a topographical map 555. For example, the SRTHD 545 may provide feedback on the patient's current neurological condition. For example, the display 515 may include features included in the Live EEG Features 405 as described with reference to FIGS. 4A-B.

FIG. 6A and FIG. 6B are schematic diagrams of an exemplary intersecting component of a RNOEAS. As shown in FIG. 6A, an intersecting module 600 (e.g., the intersecting modules 215 as described in FIGS. 2A-C) includes openings 605 on four sides. For example, the openings 605 may allow soft bands (e.g., the nylon elastic band of the supporting structure 150) to be threaded through. For example, the intersecting module 600 may releasably couple the soft bands during an operation of the RNOEAS 200. For example, the intersecting module 600 may advantageously provide resizability of the RNOEAS 200 to fit different head sizes.

FIG. 7A, FIG. 7B, and FIG. 7C are schematic diagrams of an exemplary multi-pin electrode. In this example, a multi-pin electrode 700. As shown in FIGS. 7A-C, the dry electrodes may be fabricated by a multi-pin configuration. For example, the multi-pin electrode 700 may be composed of polylactic acid (PLA). For example, the multi-pin electrode 700 may be composed of thermoplastic matrix. For example, the multi-pin electrode 700 may include, on its surface, a conductive coating 705. For example, the conductive coating may include an Ag/AgCl ink.

Each electrode may, for example, include an electrode body 704. The electrode body may, for example, be radiotranslucent. The electrode body 704 may, for example, be non-conductive.

One or more surfaces (e.g., external surface) of the electrode body may, for example, be at least partially covered with the coating 705. When the electrode 700 is operated in a recording mode, a distal end of the electrode body 704 may be in contact with the patient's skin. The coating 705 may be disposed over the surface of the electrode body such that the coating 705 provides a communication path from the patient's skin to an conductor (e.g., lead 230) at and/or near a proximal end of the electrode body 704.

The coating 704 may, for example, be applied at a thickness such that the coating 704 is effectively radiotranslucent. For example, bodies behind the electrode 700 may be readily visualized in a radiographic image (e.g., fluoroscopy image, CT image, X-ray image) through multiple (e.g., 2, 3, 4) layers of the coating 704.

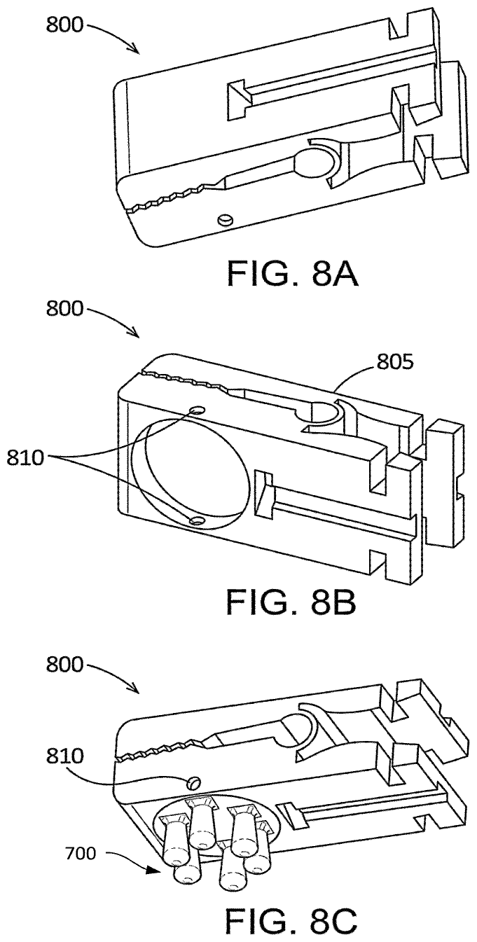

In some implementations, the multi-pin electrode 700 may include prolonged pins 710. In this example, the multi-pin electrode 700 includes 6 pins. In some implementations, other numbers of pins may be used. For example, the multi-pin electrode 700 may include 3 pins. For example, the multi-pin electrode 700 may include 5 pins. For example, the multi-pin electrode 700 may include 7 pins. For example, the multi-pin electrode 700 may include 10 pins. For example, the pins may include varying lengths. For example, the prolonged pins 710 may be longer (e.g., at 6 mm, at 4 mm, at 8 mm) for use in hairier portions of the patient's head. In some implementations, the multi-pin electrode 700 may have no pin at some portion of the head. For example, an ear clip electrode may include a cylinder-shaped pin as described with reference to FIGS. 8A-C. In some implementations, the prolonged pins 710 may be spring loaded 715 to advantageously maintain a firm contact between the electrode unit 260 and a scalp of the patient 115.

In some implementations, the Ag/AgCl coating may advantageously provide a high biocompatibility and a low contact noise. In some implementations, the conductive coating may include a carbon paint. For example, the carbon paint may advantageously be more radiotranslucent. In some implementations, the conductive coating may include a silver paint. For example, the silver paint may include better conductive properties to advantageously improve signal strength. In some implementations, a body of the multi-pin electrode 700 may include a thermoplastic polyurethane (TPU). For example, the TPU may be softer than PLA. For example, using the TPU, the multi-pin electrode 700 may advantageously be more comfortable for the patient.

FIG. 8A, FIG. 8B, and FIG. 8C are schematic diagrams of an exemplary electrode clip. In this example, an electrode clip 800 may include a housing 805. For example, the housing may include a coupling feature 810 configured to releasably couple the multi-pin electrode 700 to the supporting structure 150 of the EEG cap 120 (as described with reference to FIG. 2A). For example, the electrode clip 800 may include a rubber band threaded through and tied around to the end of the clip. For example, the housing 805 may include radiotranslucent materials.

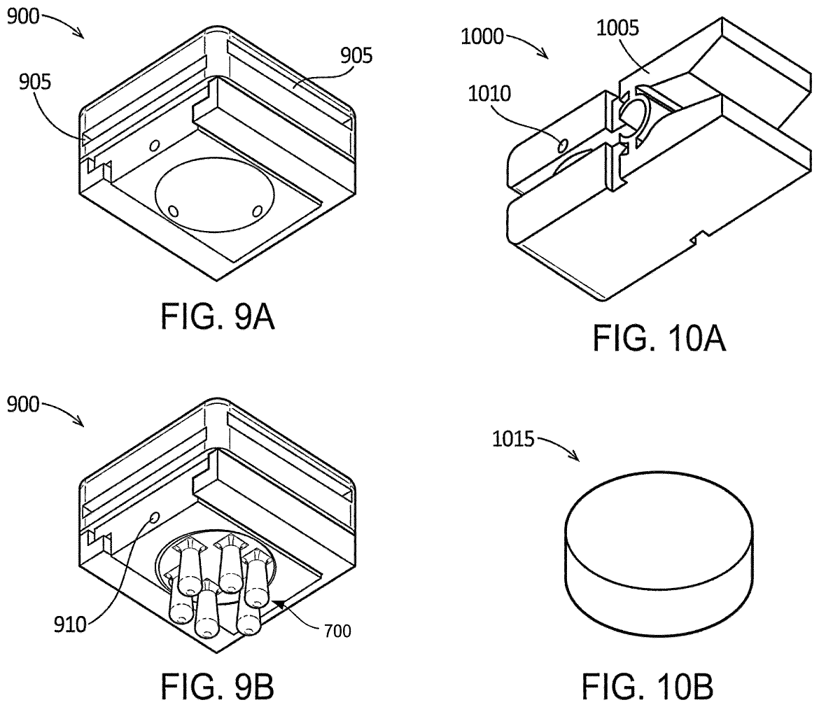

FIG. 9A and FIG. 9B are schematic diagrams of an exemplary central electrode. In some implementations, an electrode module 900 may be placed at central positions of a scalp (e.g., Fz, Cz, Pz). As shown, the electrode module 900 includes openings 905. For example, the electrode module 900 may be coupled to the bands of the EEG cap 120 using a sliding mechanism through the openings 905.

As shown in FIG. 9B, the electrode module 900 also includes a coupling feature 910. The electrode module 900 may releasably couple the intersecting module 600 to the band of the EEG cap 120 using the coupling feature 910.

FIG. 10A and FIG. 10B are schematic diagrams of an exemplary ear electrode. As shown in FIG. 10A, an ear electrode 1000 includes a clip body 1005 like the electrode clip 800. In this example, the ear electrode 1000 includes coupling feature 1010. FIG. 10B shows an exemplary electrode 1015 for the ear electrode 1000. For example, the exemplary electrode 1015 may be without pins. For example, the exemplary electrode 1015 may be releasably attached to the clip body 1005 via the coupling feature 1010.

FIG. 11A and FIG. 11B depict exemplary displays illustrating an exemplary configuration mode and an operation mode of an exemplary RNOEAS. FIG. 11A shows an exemplary display 1100 in a configuration mode of the RNOEAS 200. For example, the RVE 330 may, during the configuration mode of the RNOEAS 200, display the display 1100 to aid a user to property configure (e.g., resize, adjust location of electrodes on a patient) the RNOEAS 200.

In some implementations, the RVE 330 may listen for a data stream from the RNOEAS 200. For example, the RVE 330 may generate a visual map of individual skin-to-electrode impedances. The impedance map, as shown in display 1100, may provide information about whether each electrode is making proper contact with the skin of the patient. For example, each labelled electrode may be represented by a small circle that is colored green (as denoted by a shaded dot on the display 1100) if the measured impedance is below a threshold, and colored red (as denoted by a darker dot on the display 1100) if the impedance is more than the threshold. For example, the threshold value may be adjustable by a user (e.g., an administrative user).

As shown in FIG. 11B, a temporally distributed PSD for live EEG signals is shown. In this example, the EFG 345 may compute PSD for a time window. As shown, an x axis is time, and a y axis is frequency of the EEG signal. For example, an intensity of the displayed color may represent power. In some implementations, the BHPE 340 may use the computed PSD to search for a change in power in the time domain. For example, the BHPE 340 may look for the change in power in specific frequency bands (e.g., along the y-axis) over time (e.g., along the x-axis). For example, the BHPE 340 may determine an absence in glycolysis when a change in power in a certain frequency band is detected.

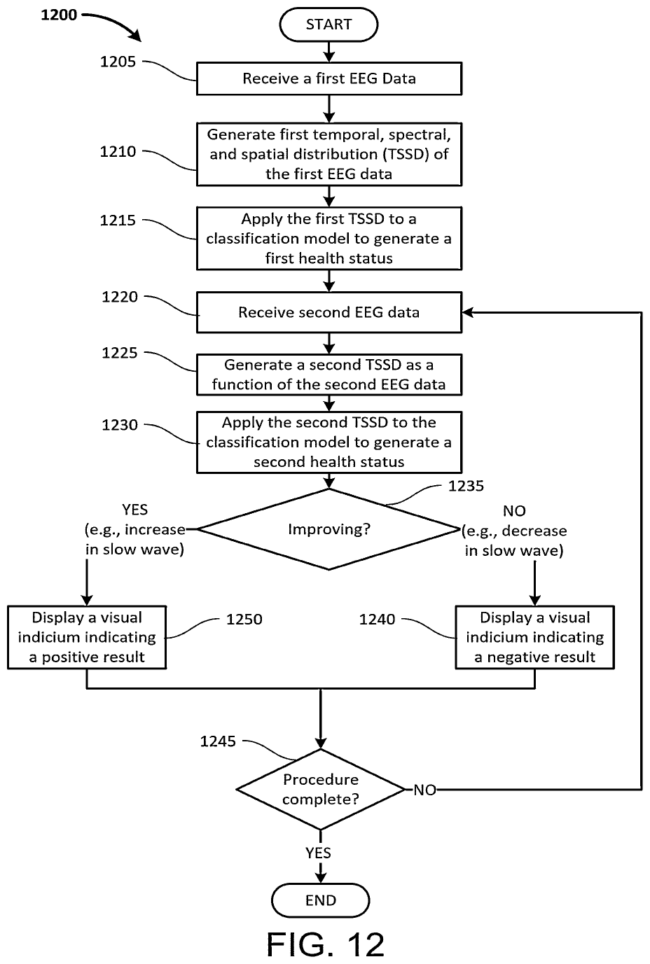

FIG. 12 is a flowchart illustrating an exemplary EEG signal monitoring method 1200. For example, the penumbra monitoring method 1200 may be performed by the BHPE 340 to monitor a brain health of a patient in an emergency procedure. The EEG signal monitoring method 1200 begins, in step 1205, when a first EEG data is received. The first EEG data may, for example, be received from the EEG cap 120.

Next, a first temporal, spectral and/or spatial distribution (TSSD) of the first EEG data is generated in step 1210. For example, the EFG 345 may generate the TSSD. In step 1215, the first TSSD is applied to a classification model to generate a first health score. For example, the Live EEG Features 405 is applied to the BHCM 350 to generate the health status matrix 410.

In step 1220, a second EEG PSD data is received. For example, the FEMS 100 may receive a second set of EEG signals after some time from the RNOEAS 200. In step 1225, a second TSSD is generated. The second TSSD is applied to the classification model to generate a second health status in step 1230. After the second health status is generated, in a decision point 1235, it is determined whether the health status is improving.

In some implementations, by way of example and not limitation, a trend may be checked multiple times (e.g., multiple time-separated second EEG data may be taken, such as real-time monitoring as an operation is in progress). For example, a current difference between the first EEG data and a current second EEG data may be compared to a previous difference between the first EEG data and a previous second EEG data. A positive trend may, for example, be determined if the second difference is larger than the first difference in a positive direction.

In some implementations, by way of example and not limitation, a trend may be checked multiple times against a target (e.g., a set of qEEG modified to compare to a target value, such as a baseline and/or a dataset corresponding to a baseline). An abnormality may be determined based on a difference between the current and target TSSD, for example.

If it is determined that the difference does not indicate an improvement in the decision point 1235, a visual indicium indicating a negative result is displayed in a step 1240 and the method 1200 proceeds to a decision point 1245 to determine if the procedure is complete (e.g., based on an input from a physician). If it is determined, in the decision point 1235, that the difference corresponds to an improvement, a visual indicium indicating a positive result is displayed in a step 1250 and the method 1200 proceeds to the decision point 1235. If it is determined, in the decision point 1245, that the procedure is complete, then the method 1200 ends. Otherwise, the method 1200 returns to the step 1225 to receive an (updated) second EEG data.

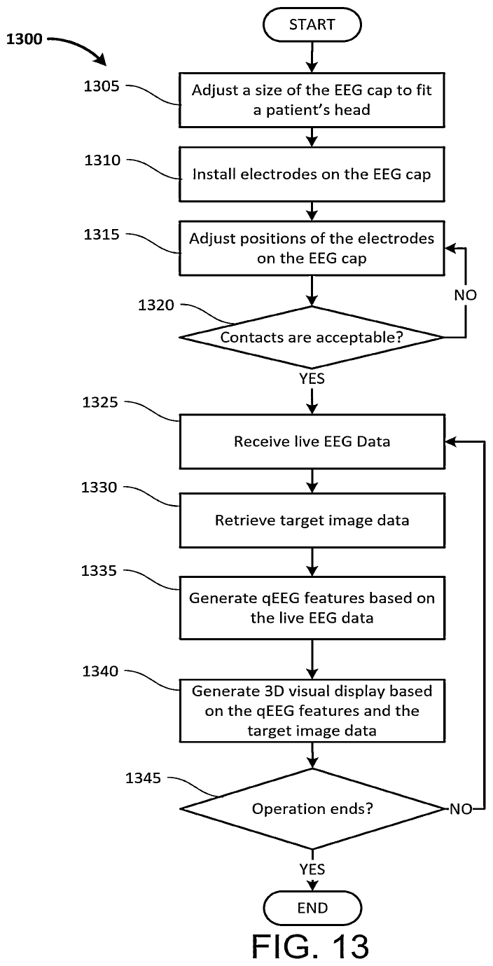

FIG. 13 is a flowchart illustrating an exemplary live brain activity visualization method 1300. For example, the live brain activity visual generation method 1300 may be performed by the RVE 330. The live brain activity visual generation method 1300 begins when a size of the EEG cap is adjusted to fit a patient's head in step 1305. For example, the EEG cap 120 may include the fabric bands 160 and the chin strap 225 that are adjustable to fit a head of the patient 115. In step 1310, electrodes are installed on the EEG cap. For example, the electrodes 155 may be installed to the supporting structure 150 by clipping on the fabric bands 160. For example, the electrodes 155 may be installed to the supporting structure 150 by threaded through at least one of the fabric bands 160 using the electrode module 900. Positions of the electrodes are adjusted on the EEG cap in step 1315. For example, the electrode module 255 may be freely adjusted along the fabric bands 160 of the EEG cap 120. In a decision point 1320, it is determined whether the contacts of the electrodes are acceptable. For example, an impedance value of each electrode may be checked using the display 1100. If any of the contacts of the electrodes is not acceptable, the step 1315 is repeated.

If the contacts of the electrodes are acceptable, live EEG data is received in step 1325. For example, the FEMS 100 may receive the EEG signals from the RNOEAS 200 via the EEG control unit 205. Next, a target image data is retrieved in step 1330. For example, the target image data may be retrieved from the other peripheral devices 315 (e.g., a data store, a fluoroscopy input module 240). In step 1335, qEEG features are generated based on the live EEG data. For example, the EFG 345 may generate qEEG features in temporal, spatial, and/or spectral domains.

In step 1340, a 3D visual display is generated based on the qEEG features and the target image data. For example, heatmaps of brain activity (e.g., the spatially distributed heatmap 520, the beta band heatmap 525, the delta band heatmap 530, the theta band heatmap 535, the time-domain health level 540) may be generated. Next, it is determined whether the operation is ended in step 1345. If it is determined that the operation is not ended, then the method 1300 repeats the step 1325. If it is determined that the operation is ended, then the method 1300 ends.

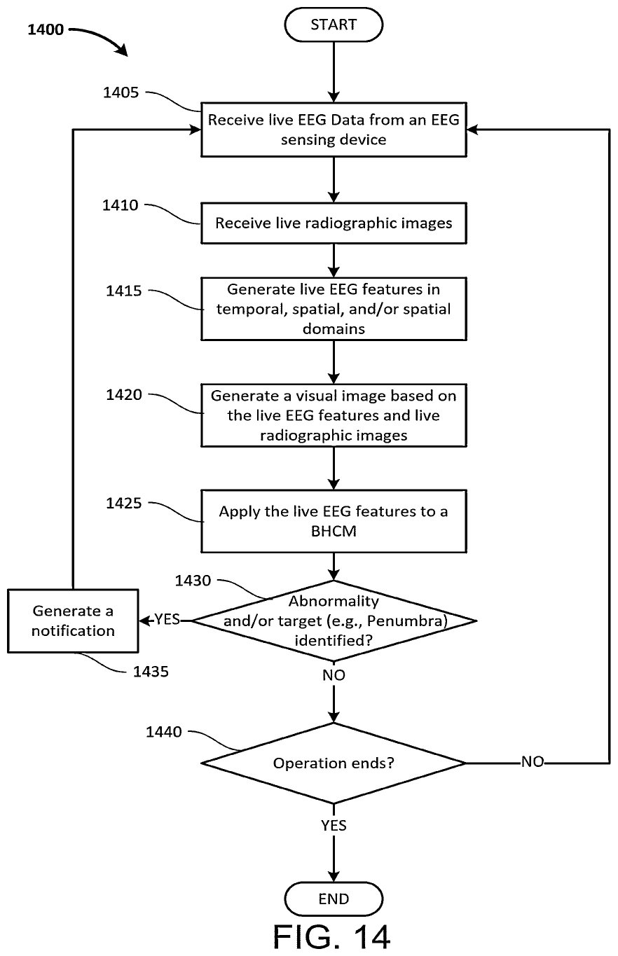

FIG. 14 is a flowchart illustrating an exemplary abnormality identification method 1400. For example, the abnormality identification method 1400 may be performed by the BHPE 340. The abnormality identification method 1400 begins when live EEG data is received from an operation in step 1405. For example, the BHPE 340 may receive measurements from the EEG cap 120 during a cardiac or vascular surgery. In some implementations, the live EEG data may be processed measurements data received from the EEG cap 120 and processed by the EEG control unit 205 and the EFG 345. In step 1410, live radiographic images are received. For example, the BHPE 340 may receive live fluoroscopy image data from the fluoroscopy input module 240.

In step 1415, live EEG features in temporal, spatial, and/or spectral domains are generated. For example, the EFG 345 may generate the EEG features as a function of the received EEG signals in temporal, spatial, and/or spectral domains. Next, a visual image is generated based on the live EEG features and the live radiographic images in step 1420. For example, the IOE 335 may generate a visual image using the live EEG PSD features and live fluoroscopy image data.

After the visual image is generated, the live EEG features are applied to a BHCM in step 1425. For example, the BHPE 340 may apply the live EEG features to the BHCM 350 to generate the health status matrix 410. In step 1430, it is determined whether any abnormality (e.g., in the penumbra) is identified. If it is determined that an abnormality is identified, then, in step 1435, a notification is generated. For example, a sound alert may be generated or a visual alert at the display module 130 (e.g., a monitor) may be displayed. Next, the method 1400 repeats the step 1405.

If it is determined that an abnormality is not identified, then it is determined whether the operation is ended in step 1440. If it is determined that the operation is not ended, then the method 1400 repeats the step 1405. If it is determined that the operation is ended, then the method 1400 ends.

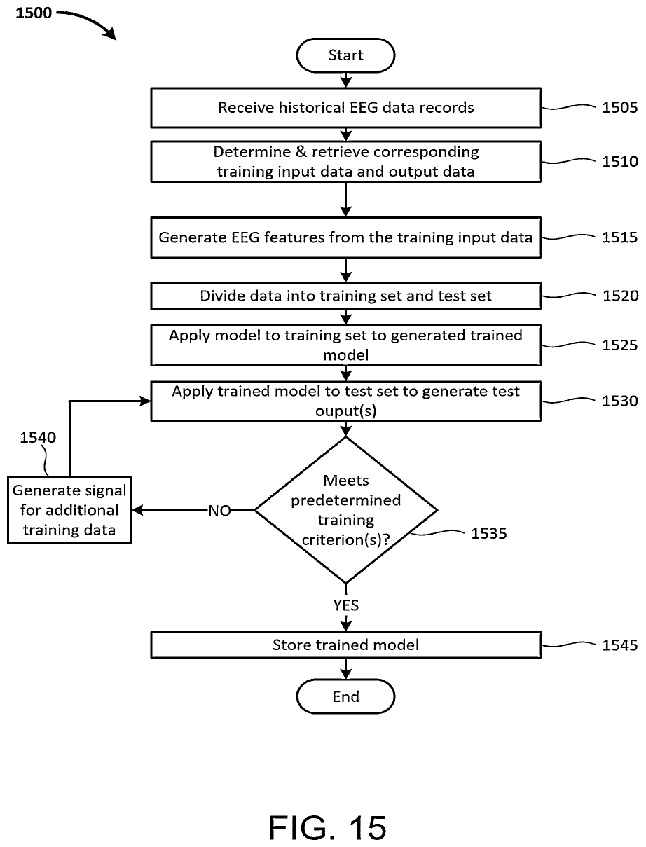

FIG. 15 depicts an exemplary method 1500 of training a classification model in an BHCM. For example, the machine learning engine 415 may execute the method 1500 to train the BHCM 350 (e.g., a DRF, a feed forward neural network). The method 1500 includes, at a step 1505, receiving historical EEG signal records. For example, the historical EEG signal records may include the anesthesia patient data set 365. At a step 1510, corresponding training input and output data (e.g., the positive and negative classification labels corresponding to each of input of the anesthesia patient data set 365) are determined and retrieved. In step 1515, EEG features are generated from the training input data. For example, the EFG 345 may be used to generate the EEG features. For example, the EEG features may include features in the spatial domain, the temporal domain, and the spectral domain.

At a step 1520, the retrieved data is divided into a first set of data used for training and a second set of data used for testing. For example, the division may be specified by the classification models parameters 430. At a step 1525, a model is applied to the training data to generate a trained model (e.g., a DRF, a neural network model). The trained model is applied to the testing data, in a step 1530, to generate test output(s) (e.g., stroke predictions). The output is evaluated, in a decision point 1535, to determine whether the model is successfully trained (e.g., by comparison to a predetermined training criterion(s)). The predetermined training criterion(s) may, for example, be a maximum error threshold. The predetermined training criterion(s) may be maximum value of a cost function of sensitivity and specificity of the output. For example, if a difference between the actual output (the test data) and the predicted output (the test output) is within a predetermined range, then the model may be regarded as successfully trained. If the difference is not within the predetermined range, then the model may be regarded as not successfully trained. At a step 1540, the processor may generate a signal(s) requesting additional training data, and the method 1500 loops back to step 1530. If the model is determined, at the decision point 1535, to be successfully trained, then the trained model may be stored (e.g., in the data store 355), in a step 1545, and the method 1500 ends.

Although various embodiments have been described with reference to the figures, other embodiments are possible. In some implementations, the fluoroscopy equipment 125 may include MRI devices.

In some implementations, the FEMS 100 may be used to identify penumbra. The penumbra may, for example, be identified based on PSD thresholds and/or comparisons (e.g., a transition region having a (predetermined) range of intensity and located between a higher intensity region (e.g., ‘healthy’ tissue) and a lower intensity region (e.g., ‘dead’ tissue). In some implementations, by way of example and not limitation, a EEG PSD data may, for example, be a historical baseline (e.g., individual, aggregated, averaged). The first EEG PSD data may, for example, correspond to a normal baseline. In some implementations, the first EEG PSD data may, for example, correspond to a disease baseline. For example, if a second EEG PSD data, generated after the first EEG PSD data, corresponds to an increase in a slow wave of the EEG in the penumbra region, the FEMS 100 may determine that the difference indicates a positive trend, indicating that a patient's situation is improving. For example, if the second EEG PSD data corresponds to a decrease in a slow wave of the EEG in the penumbra region, it may be determined, as depicted, that the difference indicates a negative trend, indicating that a patient's situation is worsening.

Although an exemplary system has been described with reference to FIGS. 1-3, other implementations may be deployed in other industrial, scientific, medical, commercial, and/or residential applications.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In various implementations, real-time may refer to delivery of a visual indicia within an amount of time realistically usable for an intended purpose. For example, real-time display of brain health indicia may, for example, refer to intraoperative display of the brain health indicia based on signal acquired during the operation. For example, real-time acquisition, processing, and/or display may advantageously solve a problem of interrupting a procedure (e.g., surgery), bringing a patient out of anesthesia, and measuring brain health indicia by EEG.

In some implementations, for example, real-time may be under 3 hours. For example, real-time may be less than 1 hour. For example, real-time may be less than 30 minutes. For example, real-time may be less than 15 minutes. In some implementations, for example, real-time may be less than 10 minutes. As an illustrative example, real-time may be less than 5 minutes. Some implementations may, for example, provide real-time results (e.g., from acquisition to display) in less than 1 minute.

In some implementations, electrodes disclosed herein may be configured for EEG.

In some implementations, electrodes disclosed herein may be configured and/or used for other electropotential measurement. For example, some electrodes may be configured for electrocardiogram (ECG). Some electrodes may, for example, be configured for measuring electropotential of non-cardiac musculature. Some electrodes may, for example, be configured to determine peripheral voluntary and/or involuntary tissue activity and/or function.

In some implementations, a electropotential measurement system may include electrodes in a predetermined placement harness (e.g., the EEG cap 120). In some implementations, for example, the harness may be configured for other body portions (e.g., heart, limb). The harness may, for example, include elastic bands. The elastic bands may, for example, be adjustably interconnected in a lattice formation such as disclosed at least with reference to the EEG cap 120.

In some implementations described herein, the EEG cap 120 may, for example, be equipped with dry electrodes. Some implementations may, for example, be equipped with wet electrodes. Some implementations may, for example, be configured as a hybrid system with dry and wet electrodes.

In some implementations described herein, one or more of the electrodes disclosed herein (e.g., electrode 700) may be configured specifically as dry electrodes. Some implementations may, for example, be implemented as wet electrodes (e.g., compatible with external conductive gel). For example, some such configurations may have flat electrodes and/or be operable in the absence of spring-loaded elements.

As an illustrative example, in a fluoroscopy environment, the EEG cap 120 may be configured with one or more wet electrodes. In some implementations, for example, a concurrent electropotential and other modality (e.g., radiography) monitoring system (e.g., a ‘hybrid’ system) may advantageously use wet and/or dry electrodes. In an illustrative aspect, a signal acquisition cap may include a soft support structure. The soft support structure may include multiple elastic bands in a lattice formation and in radiotranslucent materials. The signal acquisition cap may include a communication system. The communication system may include a network of conductive leads.

The signal acquisition cap may include a clip-on electrode unit releasably coupled to the soft support structure and electrically connected to the communication system. For example, the clip-on electrode unit may include an electrode body. The electrode body may include radiotranslucent materials. The clip-on electrode may include a conductive coating disposed on a surface of the electrode body, and a spring loaded recording channel of dry electrode. For example, the spring loaded recording channel may be configured to conduct EEG signals to the communication system through the network of conductive leads.

For example, the clip-on electrode unit may include effectively radiotranslucent materials. For example, when the clip-on electrode unit may be deployed on a skin surface, the spring loaded recording channel may be configured to conduct EEG signals from the skin surface to the communication system. For example, the EEG signals may be continuously recorded from the spring loaded recording channel without obstructing a view of a concurrently operating radioactivity imaging tool.

For example, the soft support structure may include a chin strap coupled to at least one of the elastic bands, and a band adjusting module configured to adjust a size of the soft support structure. For example, the soft support structure may be adjustable to fit different head sizes.

For example, the soft support structure may include an intersecting component configured to releasably hold two or more elastic bands at an intersection. For example, the communication system may include an amplifier circuit. For example, the amplifier circuit may be placed away from the soft support structure such that an obstruction of the view of a concurrently operating radioactivity imaging tool by the amplifier circuit may be prevented.

For example, the electrode body may include non-conducting materials. For example, the conductive coating may include a silver/silver chloride coating. For example, the network of conducting leads may include copper. For example, each lead of the network of conducting leads may include a diameter of less than 50 AWG.

For example, the clip-on electrode unit may include at least six spring loaded recording channels. The signal acquisition cap may include a threaded through dry electrode. The threaded through dry electrode may include four openings. For example, the threaded through dry electrode may be configured to be releasably coupled to the soft support structure by at least two of the elastic bands.

For example, the signal acquisition cap may include a remote computer system and a display module. For example, the remote computer system may be configured to generate temporal distributed, spatially distributed, and power spectrum density distributed EEG features as a function of the EEG signals. For example, a visualization of the EEG features may be displayed in real time at the display module.

In an illustrative aspect, a dry physiological electropotential signal acquisition electrode may include a radiotranslucent electrode body extending outward from a dry electrode unit. The electrode may include a conductive coating disposed on a surface of the electrode body extending from a proximal end of the radiotranslucent electrode body to a conductor operably coupling the electrode unit 700 to a receiver. The radiotranslucent electrode body and the conductive coating may, for example, be configured such that, when the proximal end of the radiotranslucent electrode body is deployed against a body surface the conductive coating provides signal communication from the body surface to the conductor operably coupled to the receiver, and a radiographic view of objects underlying the body surface remains simultaneously obtainable through the conductive coating and the radiotranslucent electrode body.

For example, the electrode body may include a clip.

For example, the electrode body may be coupled to an adjustable elastic headband.

For example, the electrode may include an urging member configured to urge the electrode body away from the electrode unit against the body surface.

For example, the electrode body may include non-conducting materials.

For example, the conductive coating may include a silver/silver chloride coating.

For example, the electrode unit may be configured as an electroencephalogram (EEG) electrode.

In an illustrative aspect, a signal visualization system may include a radiotranslucent electrode cap configured to detect EEG signals. The radiotranslucent electrode cap may for example, be effectively radiotranslucent in a radiographic image.

For example, the signal visualization system may include a computer system operably coupled to the radiotranslucent electrode cap to receive the EEG signals. The computer system may include an EEG features generation engine configured to generate EEG features as a function of the EEG signals. The EEG features may include features in a temporal domain, spectral domain, and spatial domain. The computer system may include a brain health classification model operably coupled to the EEG features generation engine and configured to generate, as a function of the EEG features, predetermined brain health indicia. The computer may include a display engine operably coupled to the brain health classification model and configured to generate, from the predetermined brain health indicia, at least one visual indicia including a temporally distributed, a spatially distributed, and a spectrally distributed display of the brain health indicia in near real time.

In an illustrative aspect, a signal visualization system may include a computer system operably coupled to electrodes disposed to receive electroencephalogram (EEG) signals. The computer system may include an EEG features generation engine configured to generate EEG features as a function of the EEG signals. The EEG features may include features in a temporal domain, spectral domain, and spatial domain. The computer system may include a brain health classification model operably coupled to the EEG features generation engine and configured to generate, as a function of the EEG features, real-time brain health indicia. The computer system may include a display engine operably coupled to the brain health classification model and configured to generate, from the real-time brain health indicia, at least one real-time visual indicia including a temporally distributed, a spatially distributed, and a spectrally distributed real-time display of the real-time brain health indicia.

For example, the signal visualization system may include a radiotranslucent electrode cap configured to detect EEG signals. The radiotranslucent electrode cap may, for example, be effectively radiotranslucent in a radiographic image.

For example, the real-time visual indicia include a detection of a change in power in a specific frequency band over time. For example, an absence in glycolys may be in a brain of the patient may be detected.

For example, the radiotranslucent electrode cap and the computer system may be wirelessly coupled.

For example, the radiotranslucent electrode cap may include a clip-on electrode unit. For example, the clip-on electrode unit may include an electrode body may include radiotranslucent materials, a conductive coating disposed on a surface of the electrode body, and a recording channel of dry electrode configured to conduct the EEG signals to the computer system through a conductor. For example, the clip-on electrode unit may include effectively radiotranslucent materials.

For example, in operation, the recording channel may be disposed on a skin surface of the body part and may be configured to conduct EEG signals from the skin surface to the computer system. For example, a view of a concurrently operating radioactivity imaging tool may be unobstructed.

For example, the signal visualization system may include a radiographic imaging input configured to concurrently receive an image signal of the patient under anesthesia. The radiotranslucent electrode cap may, for example, be radiotranslucent such that a view of the image signal is effectively unobstructed by the radiotranslucent electrode cap.

For example, the display engine may be configured to generate a simultaneous display including the real-time display and an image received from the radiographic imaging input. The simultaneous display may include an overlay of the real-time brain health indicia and an image received from the radiographic imaging input. The simultaneous display may include a three-dimensional visualization of a body part concurrently monitored by the radiotranslucent electrode cap and the radiographic imaging input.

For example, the brain health classification model may include classification parameters trained with anesthesia specific health conditions data set.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

1. A signal acquisition cap comprising:
a soft support structure (150) comprising a plurality of elastic bands (160) in a lattice formation and in radiotranslucent materials;
a communication system (205) comprises a network of conductive leads; and,
a clip-on electrode unit (250) releasably coupled to the soft support structure and electrically connected to the communication system, wherein the clip-on electrode unit comprises:
an electrode body (805) comprising radiotranslucent materials;
a conductive coating (705) disposed on a surface of the electrode body; and,
a spring-loaded recording channel of dry electrode (710), wherein the spring loaded recording channel comprises a multi-pin electrode (700), and is configured to conduct EEG signals to the communication system through the network of conductive leads, wherein the clip-on electrode unit comprises effectively radiotranslucent materials,
wherein, when the clip-on electrode unit is deployed on a skin surface, the spring-loaded recording channel is configured to conduct EEG signals from the skin surface to the communication system, such that
the EEG signals are continuously recorded from the spring-loaded recording channel without obstructing a view of a concurrently operating radioactivity imaging tool.

2. The signal acquisition cap of any of claim 1 or 32, wherein the soft support structure comprises:
a chin strap (225) coupled to at least one of the plurality of elastic bands; and
a band adjusting module (220) configured to adjust a size of the soft support structure, such that the soft support structure is adjustable to fit different head sizes.

3. The signal acquisition cap of any of claim 1 or 32, wherein the soft support structure further comprises an intersecting component (215) configured to releasably hold two or more elastic bands at an intersection.

4. The signal acquisition cap of any of claim 1 or 32, wherein the communication system further comprises an amplifier circuit (135), wherein the amplifier circuit is placed away from the soft support structure such that an obstruction of the view of a concurrently operating radioactivity imaging tool by the amplifier circuit is prevented.

5. The signal acquisition cap of any of claim 1 or 32, wherein the electrode body comprises non-conducting materials.

6. The signal acquisition cap of any of claim 1 or 32, wherein the conductive coating comprises a silver/silver chloride coating.

7. The signal acquisition cap of any of claim 1 or 32, wherein the network of conducting leads comprises copper.

8. The signal acquisition cap of any of claim 1 or 32, wherein each lead of the network of conducting leads comprises a diameter of less than 50 AWG.

9. The signal acquisition cap of any of claim 1 or 32, wherein the clip-on electrode unit comprises at least six spring-loaded recording channels.

10. The signal acquisition cap of any of claim 1 or 32, further comprising a threaded through dry electrode comprising four openings, wherein the threaded through dry electrode is configured to be releasably coupled to the soft support structure by at least two of the plurality of elastic bands.

11. The signal acquisition cap of any of claim 1 or 32, further comprising a remote computer system and a display module (130), wherein the remote computer system is configured to generate temporally distributed, spatially distributed, and spectrally distributed EEG features as a function of the EEG signals, such that a visualization of the EEG features is displayed in real time at the display module.

12. A dry physiological electropotential signal acquisition electrode comprising:
a radiotranslucent electrode body (704) extending outward from a dry electrode unit (700)
a conductive coating (705) disposed on a surface of the radiotranslucent electrode body extending from a proximal end of the radiotranslucent electrode body to a conductor operably coupling the electrode unit 700 to a receiver,
wherein at least the radiotranslucent electrode body and the conductive coating are configured such that, when the proximal end of the radiotranslucent electrode body is deployed against a body surface:
the conductive coating provides signal communication from the body surface to the conductor operably coupled to the receiver, and
a radiographic view of objects underlying the body surface remains simultaneously obtainable through the conductive coating and the radiotranslucent electrode body.

13. The electrode of claim 12, wherein the electrode body comprises a clip (250).

14. The electrode of claim 12, wherein the electrode body is coupled to an adjustable elastic headband (150).

15. The electrode of claim 12, further comprising an urging member (715) configured to urge the electrode body away from the electrode unit against the body surface.

16. The electrode of claim 12, wherein the electrode body comprises non-conducting materials.

17. The electrode of claim 12, wherein the conductive coating comprises a silver/silver chloride coating.

18. The electrode of claim 12, wherein the electrode unit is configured as an electroencephalogram (EEG) electrode.

19. A signal visualization system comprising:
a radiotranslucent electrode cap (200) configured to detect EEG signals, wherein the radiotranslucent electrode cap is effectively radiotranslucent in a radiographic image; and,
a computer system (100) operably coupled to the radiotranslucent electrode cap to receive the EEG signals, wherein the computer system comprises:
an EEG features generation engine (345) configured to generate EEG features as a function of the EEG signals, wherein the EEG features comprises features in a temporal domain, spectral domain, and spatial domain;
a brain health classification model (350) operably coupled to the EEG features generation engine and configured to generate, as a function of the EEG features, predetermined brain health indicia; and,
a display engine (330) operably coupled to the brain health classification model and configured to generate, from the predetermined brain health indicia, at least one visual indicia comprising:
a temporally distributed, a spatially distributed, and a spectrally distributed display of the brain health indicia in near real time, wherein the spectrally distributed comprises a power spectrum density of the EEG signals; and,
an overlay of the brain health indicia and an image received from an imaging input device in near real time.

20. A signal visualization system comprising:
a computer system operably coupled to a plurality of electrodes disposed to receive electroencephalogram (EEG) signals corresponding to a patient, the computer system comprising:
an EEG features generation engine (345) configured to generate EEG features as a function of the EEG signals, wherein the EEG features comprises features in a temporal domain, spectral domain, and spatial domain;
a brain health classification model (350) operably coupled to the EEG features generation engine and configured to generate, as a function of the EEG features, real-time brain health indicia; and,
a display engine (330) operably coupled to the brain health classification model and configured to generate, from the real-time brain health indicia, at least one real-time visual indicia comprising:
a temporally distributed, a spatially distributed, and a spectrally distributed real-time display of the real-time brain health indicia, wherein the spectrally distributed comprises a power spectrum density of the EEG signals; and,
an overlay of the real-time brain health indicia and an image received from an imaging input device.

21. The signal visualization system of claim 20, further comprising a radiotranslucent electrode cap (200) configured to detect EEG signals, wherein the radiotranslucent electrode cap is effectively radiotranslucent in a radiographic image.

22. The signal visualization system of claim 21, wherein the radiotranslucent electrode cap and the computer system are wirelessly coupled.

23. The signal visualization system of claim 21, wherein the radiotranslucent electrode cap comprises a clip-on electrode unit (700), wherein the clip-on electrode unit comprises:
an electrode body comprising radiotranslucent materials;
a conductive coating disposed on a surface of the electrode body; and,
a recording channel of dry electrode configured to conduct the EEG signals to the computer system through a conductor, wherein the clip-on electrode unit comprises effectively radiotranslucent materials, such that,
in operation, the recording channel is disposed on a skin surface of a body part and is configured to conduct EEG signals from the skin surface to the computer system, such that a view of a concurrently operating radioactivity imaging tool is unobstructed.

24. The signal visualization system of claim 21, wherein the imaging input device comprises a radiographic imaging input configured to concurrently receive an image signal of the patient under anesthesia, wherein the radiotranslucent electrode cap is radiotranslucent such that a view of the image signal is effectively unobstructed by the radiotranslucent electrode cap.

25. The signal visualization system of claim 24, wherein the display engine is configured to generate a simultaneous display comprising the real-time display and an image received from the radiographic imaging input.

26. (canceled)

27. The signal visualization system of claim 25, wherein the simultaneous display comprises a three-dimensional visualization of a body part concurrently monitored by the radiotranslucent electrode cap and the radiographic imaging input.

28. The signal visualization system of claim 20, wherein the brain health classification model comprises classification parameters trained with anesthesia specific health conditions data set.

29. The signal visualization system of claim 20, wherein the real-time display comprises a detection of a change in power in specific frequency band over time, such that an absence in glycolysis in a brain of the patient is detected.

30. (canceled)

31. (canceled)

32. A signal acquisition cap comprising:
a soft support structure (150) comprising a plurality of elastic bands (160) in a lattice formation and in radiotranslucent materials;
a communication system (205) comprises a network of conductive leads; and,
a clip-on electrode unit (250) releasably coupled to the soft support structure and electrically connected to the communication system, wherein the clip-on electrode unit comprises:
an electrode body (805) comprising radiotranslucent materials;
a conductive coating (705) disposed on a surface of the electrode body; and,
a spring-loaded recording channel comprises a pin (710) spring-loaded (715) and prolonged from a surface of the electrode body, wherein the spring-loaded pin comprises a spring-loaded recording channel is configured to conduct EEG signals to the communication system through the network of conductive leads, wherein the clip-on electrode unit comprises effectively radiotranslucent materials,
wherein, when the clip-on electrode unit is deployed on a skin surface, the spring-loaded recording channel is configured to conduct EEG signals from the skin surface to the communication system, such that
the EEG signals are continuously recorded from the spring-loaded recording channel without obstructing a view of a concurrently operating radioactivity imaging tool.
